(19) United States
(12) Patent Application Publication
(10) Pub. No.: US 2021/0255196 A1
(43) Pub. Date: Aug. 19, 2021

(54) LOW BINDING SURFACES FOR PEPTIDE MAPPING

(71) Applicant: Waters Technologies Corporation, Milford, MA (US)

(72) Inventors: Robert Birdsall, Westborough, MA (US); Jacob Kellett, Whitinsville, MA (US); Nilini Ranbaduge, Hopkinton, MA (US); Ying Qing Yu, Uxbridge, MA (US); Jennifer M. Nguyen, Uxbridge, MA (US); Matthew A. Lauber, North Smithfield, RI (US)

(73) Assignee: Waters Technologies Corporation, Milford, MA (US)

(21) Appl. No.: 17/151,036

(22) Filed: Jan. 15, 2021

Related U.S. Application Data
(60) Provisional application No. 62/962,480, filed on Jan. 17, 2020.
(60) Provisional application No. 62/968,688, filed on Jan. 31, 2020.
(60) Provisional application No. 63/058,724, filed on Jul. 30, 2020.
(60) Provisional application No. 63/091,169, filed on Oct. 13, 2020.

(51) Int. Cl. G01N 33/68; G01N 30/72; B01J 20/281

(52) U.S. Cl. CPC G01N 33/6848; G01N 2030/484; G01N 30/482; G01N 30/7233

(57) ABSTRACT

The present disclosure discusses a method of separating a sample (e.g., peptide compound) including coating a flow path of a chromatographic system; injecting the sample into the chromatographic system; flowing the sample through the chromatographic system; separating the sample; and analyzing the separated sample. In some examples, the coating applied to the surfaces defining the flow path is non-binding with respect to the sample—and the separated sample. Consequently, the sample does not bind to the low-binding surface of the coating (e.g., organosilica coating) of the flow path. The applied coating can reduce peak tailing and increase analyte recovery for the sample of the chromatographic system.

### CROSS-REFERENCE TO RELATED APPLICATIONS

This application claims priority to U.S. Provisional Application No. 62/962,480 filed on Jan. 17, 2020, U.S. Provisional Application No. 62/962,688 filed on Jan. 31, 2020, U.S. Provisional application No. 63/058,724 filed on Jul. 30, 2020, and U.S. Provisional application No. 63/091,169 filed on Oct. 13, 2020, the contents of each are incorporated herein by reference in its entirety.

### SEQUENCE LISTING

The instant application contains a Sequence Listing which has been submitted electronically in ASCII format and is hereby incorporated by reference in its entirety. Said ASCII copy, created on Jan. 13, 2021, is named W-4202-US05_SL.txt and is 10,332 bytes in size.

### FIELD OF THE TECHNOLOGY

The present disclosure relates to the use of vapor deposition coated flow paths for improved chromatography and sample analysis for peptide mapping and acidic sample analysis. More specifically, this technology relates to separating analytes in a sample using chromatographic devices having coated flow paths, methods of separating analytes in a sample (for example, peptides (synthetic or natural), acidic samples) using a fluidic system that includes coated flow paths, and methods of tailoring a fluidic flow path for separation and analysis of peptides and acidic samples.

### BACKGROUND

Analytes that interact with metal have often proven to be very challenging to separate. The desire to have high pressure capable chromatographic systems with minimal dispersion has required that flow paths decrease in diameter and be able to withstand increasingly high pressures at increasingly fast flow rates. As a result, the material of choice for chromatographic flow paths is often metallic in nature. This is despite the fact that characteristics of certain analytes, for example, biomolecules, proteins, glycans, peptides, oligonucleotides, pesticides, bisphosphonic acids, anionic metabolites, and zwitterions like amino acids and neurotransmitters, are known to have unfavorable interactions, so called chromatographic secondary interactions, with metallic surfaces.

The proposed mechanism for metal specific binding interactions requires an understanding of the Lewis theory of acid-base chemistry. Pure metals and metal alloys (along with their corresponding oxide layers) have terminal metal atoms that have characteristics of a Lewis acid. More simply, these metal atoms show a propensity to accept donor electrons. This propensity is even more pronounced with any surface metal ions bearing a positive charge. Analytes with sufficient Lewis base characteristics (any substance that can donate non-bonding electrons) can potentially adsorb to these sites and thus form problematic non-covalent complexes. It is these substances that are defined as metal-interacting analytes.

For example, analytes having phosphate groups are excellent polydentate ligands capable of high affinity metal chelation. This interaction causes phosphorylated species to bind to the flow path metals thus reducing the detected amounts of such species, a particularly troublesome effect given that phosphorylated species are frequently the most important analytes of an assay.

Other characteristics of analytes can likewise pose problems. For example, carboxylate groups also have the ability to chelate to metals, albeit with lower affinities than phosphate groups. Yet, carboxylate functional groups are ubiquitous in, for example, biomolecules, giving the opportunity for cumulative polydentate-based adsorptive losses or undesirable chromatographic performance. These complications can exist not only on peptides and proteins, but also glycans bearing peptides or glycopeptides.

Analytes such as peptides (synthetic or natural) intrinsically contain acidic residues in the form of carboxylate groups which can be in the form of the c-terminus of all peptides or as a part of the amino acids that comprise the peptides as in the case of glutamic acid or aspartic acid. These carboxylate groups have the ability to exhibit polydendate characteristics and chelate to metals. Given the ubiquitous nature of carboxylate groups in peptide structures, provides the opportunity for cumulative polydentate-based absorptive losses or undesirable chromatographic performance. These complications can exist not only on peptides, but also glycan bearing peptides or glycopeptides. For example, N-glycan species can at times contain one or more phosphate groups, another well-known functional group that exhibits polydendate properties, or one or more carboxylate containing sialic acid residues. In this case, the extended structure of the peptide can present regions with chemical properties that amplify a secondary interaction to the material of a flow path. This, combined with the cumulative metal chelation effects, curtails the overall effective separation of biomolecules such as peptides.

An alternative to using metal flow paths is to use flow paths constructed from polymeric materials, such as polyether ether ketone (PEEK). PEEK tubing, like most polymeric materials, is formed by means of an extrusion process. With polymeric resin, this manufacturing process can lead to highly variable internal diameters. Accordingly, PEEK column hardware yields unfavorable differences in the retention times as can be observed from switching between one column and the next. Often, this variation can be a factor of three higher than a metal constructed column. In addition, the techniques for fabricating polymer based frits are not yet sufficiently optimized to afford suitably rugged components for commercial HPLC columns. For example, commercially available PEEK frits tend to exhibit unacceptably low permeability.

Ongoing efforts to reduce chelation and secondary chromatographic interactions of analytes with metal chromatographic surfaces in an effort to facilitate chromatographic separation having higher resolutions are therefore needed. In addition, variability in the separation and detection of compounds can be caused by many factors. One such factor is analyte/surface interactions of compounds with the analytical column. Such interactions can be problematic, especially at very low concentrations of analytes. This is especially true for peptide mapping.

### SUMMARY

Secondary interaction or adsorption of metal sensitive analytes to active sites dispersed throughout the metallic surface in liquid chromatography based separations have often been challenging to separate. To address problems experienced in separations in metallic fluidic systems, column hardware using a coating has been developed to define a low-binding surface(s) (LBS). Column hardware with LBS can positively impact chromatographic performance in terms of band broadening, peak tailing, and/or recovery which in turn can increase resolution, peak capacity, and/or quantitative accuracy of liquid chromatography-based assays, and in particular liquid chromatography-based peptide mapping assays.

Recently, MS-based peptide mapping has undergone considerable evaluation in development and manufacturing environments to improve productivity and data quality by effectively monitoring multiple critical quality attributes (CQAs) simultaneously. However, MS-based peptide assays are often deployed with weaker mobile phase additives such as formic acid in favor of sensitivity over chromatographic performance. This is of concern in industry where routine assays are expected to perform with consistent and accurate results. Recent observations have shown that column and LC hardware should also be given serious consideration to improve assay reproducibility and sensitivity. Specifically, metal-ion mediated adsorption in liquid chromatography (LC) has been observed as a contributing factor to poor peak shape, tailing, and diminished recovery of sensitive analytes. By utilizing the present technology, including the coated column hardware, improvements can be achieved in assay sensitivity, recovery, and reproducibility.

In addition, for peptide mapping and acidic sample analysis sample throughput can be increased by using the technology of the present disclosure. Sample throughput can be increased by reduced peak tailing and increased resolution. For example, if impurities are closely eluting with the native peak and the native peak was exhibiting a degree of tailing, a user (e.g., an analyst) may try to extend the gradient or run-time to resolve impurities to an acceptable resolution between peaks that facilitated accurate quantitation. In the absence of tailing, a user could shorten the run time by using a steeper slope in the gradient. This could effectively elute everything faster and closer together. But the resolution between peaks, while decreasing, may still be sufficient for the assay since tailing is not present to interfere with integration or cause a co-elution. With reduced peak tailing, new trace species can be detected by being able to see peaks that were formerly covered by peak tailing.

And increased resolution or more time between peaks can allow a user to run faster methods with increased throughput. If resolution has increased, then peak capacity increases meaning more peaks can fit in the same chromatogram or a faster separation could be run at the cost of resolution and peak capacity if the critical pair of interest were resolved sufficiently to start with.

The present technology includes a coating, such as alkylsilyl coating, that can provide a LBS to reduce peak tailing and increase stability of the tailing factor from initial injection of a sample onward, increase analyte recovery, increase sensitivity, as well as reproducibility by minimizing the analyte/surface interactions that can lead to sample losses. Additionally, LBS coated hardware does not appear to adversely affect chromatographic performance or recovery of peptides. For example, as discussed herein, comparable peak widths were observed across an acidic ladder series for LBS coated surfaces. Also, similar retention times were observed for LBS coated and non-coated surfaces.

A chromatographic column incorporating the coating of the present disclosure has been designed to minimize negative analyte/surface interactions for compounds. Analytes, such as peptides (synthetic or natural) intrinsically contain acidic residues in the form of carboxylate groups, which can be in the form of the c-terminus of all peptides or as a part of the amino acids that include the peptide as in the case of glutamic acid or aspartic acid. In the present disclosure, metal sensitive compounds, such as peptides, were tested with and without the coating on the column hardware. Existing techniques to mitigate these interactions, such as system passivation with nitric acid, are time consuming and only produce temporary performance gains. It is difficult to determine when the system is fully passivated and ready to operate. If attempts are made to obtain data for quantitative studies before full passivation is reached, the lower end of the calibration curve would not be detected because the analyte still has metallic surfaces it can bind to.

An alkylsilyl coating on the surface area defining the flow path of a chromatographic system (e.g., a fluid-contacting coating covering metallic surfaces) can minimize the interactions between peptide compounds and the metallic surfaces of chromatographic flow paths. Consequently, the coated metallic surfaces improve liquid chromatography separations for peptide compounds. The use of alkylsilyl coatings on metal flow paths allows the use of metal chromatographic flow paths, which are able to withstand high pressures at fast flow rates, high pressure generated using stationary phases with small particles (which can be slow flow as well), and high pressure generated from longer column beds, while minimizing the secondary chromatographic interactions between peptide compounds and the metal. These components made of high-pressure material and modified with a coating can be tailored so that the internal flow paths reduce secondary chromatographic interactions. The coating covers the metallic surfaces that are exposed to the fluidic path (i.e., a fluid-contacting coating).

In one aspect, the present technology is directed to a method of separating and analyzing a metal-sensitive sample. The method includes injecting the metal-sensitive sample into a chromatographic system having a fluid-contacting coating on a metallic surface; flowing the metal-sensitive sample through the chromatographic system; separating the metal-sensitive sample, wherein coating the metallic flow path of the chromatographic system reduces peak tailing; and passing the separated metal-sensitive sample through a mass spectrometer to analyze the separated sample. The fluid-contacting coating can include an alkylsilyl.

The above aspect can include the following feature. In one embodiment, peak tailing is reduced by at least about 50%. In another embodiment, peak tailing is reduced by at least about 25%. In another embodiment, peak tailing is reduced by at least about 15%. In another embodiment, peak tailing is reduced by at least about 5%.

In one aspect, the present technology is directed to a method of separating a metal-sensitive sample. The method includes providing a chromatographic system having a fluid-contacting coating on at least a portion of a metallic flow path; injecting the metal-sensitive sample into the chromatographic system; flowing the metal-sensitive sample through the chromatographic system; separating the metal-sensitive sample, wherein the metal-sensitive sample comprises a peptide; and performing mass spectrometry on the separated metal-sensitive sample.

In another aspect, the present technology is directed to a method of separating a metal-sensitive sample. The method includes injecting the sample into a chromatographic system having a fluid-contacting coating on a metallic surface, wherein the fluid-contacting coating comprises an alkylsilyl; flowing the metal-sensitive sample through the chromatographic system; separating the metal-sensitive sample, wherein the metal-sensitive sample comprises a peptide; and analyzing the separated metal-sensitive sample with a UV detector.

The above aspects can include one or more of the following features. In one embodiment, the fluid-contacting coating increases recovery of the metal-sensitive sample by at least about 20%. In another embodiment, the fluid-contacting coating increases recovery of the metal-sensitive sample by at least about 15%. In another embodiment, the fluid-contacting coating increases recovery of the metal-sensitive sample by at least about 5%. In one embodiment, the fluid-contacting coating does not substantially change retention of the metal-sensitive sample. In one embodiment, the fluid-contacting coating does not introduce new peaks or remove peaks when compared to using the same method on a non-coated metallic flow path (i.e., fluid exposed metallic surfaces). In one embodiment, the fluid-contacting coating does not result in peak loss or diminish recovery of the metal sensitive sample. In one embodiment, the metal-sensitive sample does not bind to the fluid-contacting coating. In one embodiment, the metal-sensitive sample is selected from the group consisting of glutamic acid and aspartic acid. In one embodiment, the fluid-contacting coating includes bis(trichlorosilyl)ethane or bis(trimethoxysilyl)ethane. In one embodiment, the fluid-contacting coating reduces peak tailing. In one embodiment, peak tailing is reduced by at least about 50%.

The above aspects and features of the present technology provide numerous advantages over the prior art. In some embodiments, there are numerous benefits incorporating the coating on the column. For example, the present disclosure shows the benefits of reduced tailing factor and band broadening, increased analyte recovery and chromatographic stability, and no adverse chromatography performance effects.

### BRIEF DESCRIPTION OF THE DRAWINGS

The technology will be more fully understood from the following detailed description taken in conjunction with the accompanying drawings, in which:

FIG. 1 is a schematic of a chromatographic flow system including a chromatography column and various other components, in accordance with an illustrative embodiment of the technology. A fluid is carried through the chromatographic flow system with a fluidic flow path extending from a fluid manager to a detector, such as a MS detector.

FIG. 2 is a flow chart of a method of coating a fluidic path (such as a fluidic path in a chromatography system) according to an illustrative embodiment of the technology.

FIG. 3 is a flow chart showing a method of tailoring a fluidic flow path for separation of a sample including a peptide, in accordance with an illustrative embodiment of the technology.

FIG. 4A shows UV chromatogram of a peptide map of the NIST mAb digest using the method described in accordance with Table 1.

FIG. 4B shows MS Total Ion Chromatogram (TIC) of the same peptide map acquired with the in-line QDa mass detector.

FIG. 4C shows Extracted Ion Chromatogram (XIC) of the T37 “acidic” peptide from the peptide map to indicate approximate elution region and profile.

FIG. 4D shows XIC of the T14 “acidic” peptide from the peptide map to indicate approximate elution region and profile.

FIGS. 5A-5E are representative acidic ladder chromatograms for glutamic acid “E” series. Peptides were synthetically manufactured and chromatographically separated under step gradient conditions shown in Table 2 with the following sequences. FIGS. 5A-5E disclose SEQ ID NOS 4-8, respectively, in order of appearance.

FIG. 6A and FIG. 6B display how chromatographic performance was evaluated.

FIG. 7 displays the tailing factor that was evaluated for the T37: GFYPSDIAVEWESNGQPENNYK (SEQ ID NO: 1) acidic peptide (T37-PENNYK (SEQ ID NO: 2)) using the selected ion recording (SIR) function of the QDa with a m/z of 849.20 being acquired for evaluation.

FIG. 8 displays the T14 tailing factor that was evaluated for the T14: VDNALQSGNSQESVTEQDSK (SEQ ID NO: 3) acidic peptide using the selected ion recording (SIR) function of the QDa with an m/z of 713.00 being acquired for evaluation.

FIG. 9A and FIG. 9B show the reproducibility of LBS surfaces. FIG. 9A discloses “PENNYK” as SEQ ID NO: 2.

FIGS. 10A-10E show the acidic ladder, E-series, for non-coated and LBS coated surfaces.

FIG. 11A and FIG. 11B display peptide recovery and conditioning for non-coated and LBS coated surfaces. FIG. 11A discloses “PENNYK” as SEQ ID NO: 2.

FIG. 12A and FIG. 12B show the selectivity comparison of LBS-coated versus non-coated surfaces.

FIG. 13A and FIG. 13B show a peptide profile comparison.

FIG. 14 shows extracted ion chromatograms of NISTmAb tryptic peptide T14 for non-coated (top) and coated (bottom) hardware. FIG. 14 discloses SEQ ID NO: 3.

FIG. 15 shows the MS response for T14 peptide monitored using UNIFI peptide mapping workflow for three replicate LC-MS injections on coated (left side) and non-coated (right side) hardware.

FIG. 16A, FIG. 16B, and FIG. 16C (collectively referred to as FIG. 16) show annotated fragmentation spectra for T14 NISTmAb tryptic peptide that was generated by collision induced dissociation. The top spectrum (FIG. 16A) is for non-coated hardware and the bottom spectrum (FIG. 16B) is for coated hardware. FIG. 16 discloses “VDNALQSGNSQESVTEQDSK” as SEQ ID NO: 3.

FIG. 17A and FIG. 17B show data of protein sequence coverage observed for NISTmAb digest standard for non-coated (FIG. 17A) and coated (FIG. 17B). FIG. 17A and FIG. 17B disclose SEQ ID NOS 13, 13, 14, and 14, respectively, in order of appearance.

FIG. 18 shows total ion chromatograms of for three peptide samples for non-coated (top) and coated (bottom) hardware. The first sample is doubly phosphorylated insulin receptor peptide; the second is Enolase T37; and the third sample is Angiotensin I.

FIG. 19 shows spectra of doubly phosphorylated insulin receptor and illustrate the reduction in metal adducts in the coated hardware (bottom spectra) over the non-coated hardware (top spectra).

FIG. 20A, FIG. 20B, FIG. 20C, FIG. 20D, FIG. 20E, and FIG. 20F (collectively referred to as FIG. 20) show spectra of doubly phosphorylated insulin receptor and illustrate the reduction in metal adducts in the coated hardware (FIG. 20B, FIG. 20D, and FIG. 20F) over the non-coated hardware (FIG. 20A, FIG. 20C, and FIG. 20E).

FIG. 21 and FIG. 22 show spectra of Enolase T37 and illustrate the reduction in metal adducts in the coated hardware (bottom spectra) over the non-coated hardware (top spectra).

FIG. 23 and FIG. 24 show spectra of Angiotensin I and illustrate the reduction in metal adducts in the coated hardware (bottom spectra) over the non-coated hardware (top spectra).

FIG. 25A and FIG. 25B display UV chromatograms of the fourth injection (before conditioning) and fifth injection (after conditioning) of an equimolar mixture of doubly phosphorylated insulin receptor peptide (1), Angiotensin I (2), and enolase T37 (3) obtained using a standard column (FIG. 25A) or a column constructed using HBS hardware (FIG. 25B).

FIG. 26A and FIG. 26B display mass spectra of Angiotensin I from a separation of an equimolar mixture of doubly phosphorylated insulin receptor peptide (1), Angiotensin I (2) and enolase T37 (3) obtained using a previously conditioned standard column (FIG. 26A) or a column constructed using the HBS (FIG. 26B).

FIG. 27A and FIG. 27B display the accelerated stability test results for a 4.6 mm diameter 0.2 μm titanium frit with the HBS. FIG. 27A displays the pH 1 tests that used 1% TFA (aq), and FIG. 27B displays the pH 12 tests that used 10 mM NaOH (aq).

FIG. 28A and FIG. 28B display a comparison of the separation of AMP, ADP and ATP using a standard BEH C₁₈ column (FIG. 28A) and a BEH C₁₈ column constructed with hardware treated with the HBS (FIG. 28B). FIG. 28C displays a plot of recovery of each analyte vs. injection number.

FIG. 29A, FIG. 29B, FIG. 29C, and FIG. 29D show a comparison of the separation of AMP and ATP using a standard UHPLC system (FIG. 29A and FIG. 29B) and a UHPLC system constructed using parts treated with the HBS (FIG. 29C and FIG. 29D). Fifteen sequential injections of the mixture (20 ng each analyte) were made. Chromatograms are shown for injection 1 (FIG. 29A and FIG. 29C) and injection 15 (FIG. 29B and FIG. 29D) for both UHPLC systems.

FIG. 30A displays a comparison of the peak area of ATP vs. injection number using different mobile phase pH values for a standard ACQUITY™ BEH C₁₈ column and a BEH C₁₈ column constructed with hardware treated with the HBS.

FIG. 30B displays ATP recovery vs. injection number using different injection loads for a standard ACQUITY™ BEH C₁₈ column.

FIG. 31 displays a synthetic acidic peptide ladder used to evaluate tailing for 3 synthetic peptides manufactured with 0, 2, and 4 glutamic acid (E) residues representing 0%, 10%, and 20% acidic content by composition. FIG. 31 discloses SEQ ID NOS 4, 6, and 8, respectively, in order of appearance.

FIG. 32A and FIG. 32B display recovery of a T37 peptide fragment from a tryptic digest of the NIST reference mAb standard that was evaluated for a peptide map performed on a conventional column (stainless-steel; FIG. 32A) as well as a column incorporating LBS coating technology (FIG. 32B). FIG. 32C and FIG. 32D display a 4-fold increase in peak area (FIG. 32C) and a 10-fold increase in detector response (FIG. 32D) for the conventional column (stainless-steel) versus the column incorporating LBS coating technology.

FIG. 33 displays a phosphopeptide application to demonstrate the performance differences between a peptide column including LBS coating technology and a commercially available column (non-coated column).

FIG. 34A and FIG. 34B display a comparison of the chromatographic performance of a peptide C18 column with coating technology in accordance with the present technology versus a titanium-lined C18 column technology.

FIG. 35 displays the structure of Angiotensin I.

### DETAILED DESCRIPTION

In general, the present disclosure is related to coating columns to have low-binding surfaces (LBS) to increase analyte recovery, reproducibility and sensitivity by minimizing negative analyte/surface interactions that can lead to sample losses. The present disclosure addresses the problematic binding of peptide compounds on metallic surfaces of chromatographic systems. For example, peptide compounds can interact with stainless steel to reduce analyte recovery and that this interaction can increase with the number of carboxylate groups present.

In addition, coating the system to have LBS minimizes uncertainty of the chromatographic system performance. Permanent passivation (or at least semi-permanent passivation, i.e., useable lifetime of a consumable) can be provided by the coating. For example, the system does not need to be passivated after each wash, and passivation does not effectively diminish after each wash or flowing. Consequently, the analyte detected using LC and a detector (e.g., MS, UV (for abundant species), etc.) can be depended upon as an accurate assessment of the analyte present.

One method of coating for LBS is the use of alkylsilyl coatings. In some aspects, the alkylsilyl coating acts a bioinert, low-bind coating to modify a flow path to address flow path interactions with an analyte, such as a metal-sensitive analyte. That is, the bioinert, low-bind coating minimizes surface reactions with the metal interacting compounds and allows the sample to pass along a flow path without clogging, attaching to surfaces, or change in analyte properties. The reduction/elimination of these interactions is advantageous because it allows for accurate quantification and analysis of a sample containing peptide compounds or other metal-sensitive compounds. The coating which creates LBS along the flow path prevents/significantly minimizes analyte loss to the metallic surface walls, thereby reducing secondary chromatographic interactions.

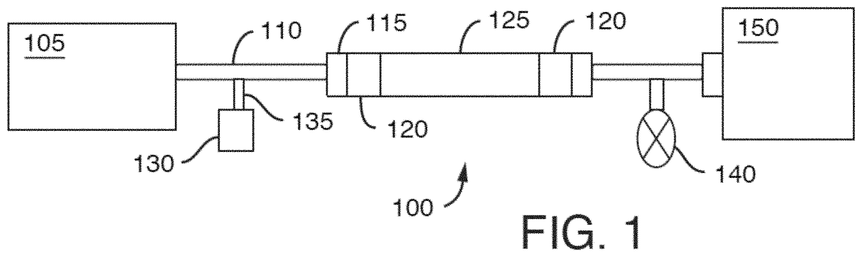

FIG. 1 is a representative schematic of a chromatographic flow system/device 100 that can be used to separate analytes, such as peptide compounds, in a sample. Chromatographic flow system 100 includes several components including a fluid manager system 105 (e.g., controls mobile phase flow through the system), tubing 110 (which could also be replaced or used together with micro fabricated fluid conduits), fluid connectors 115 (e.g., fluidic caps), frits 120, a chromatography column 125, a sample injector 135 including a needle (not shown) to insert or inject the sample into the mobile phase, a vial, sinker, or sample reservoir 130 for holding the sample prior to injection, and a detector 150, such as a mass spectrometer. Interior surfaces of the components of the chromatographic system/device form a fluidic flow path that has wetted surfaces. The fluidic flow path can have a length to diameter ratio of at least 20, at least 25, at least 30, at least 35 or at least 40.

At least a portion of the wetted surfaces can be LBS by coating with an alkylsilyl coating to reduce secondary interactions by tailoring hydrophobicity. The coating can be applied by vapor deposition. As such, methods and devices of the present technology provide the advantage of being able to use high pressure resistant materials (e.g., stainless steel) for the creation of the flow system, but also being able to tailor the wetted surfaces of the fluidic flow path to provide the appropriate hydrophobicity so deleterious interactions or undesirable chemical effects on the sample can be minimized. In some examples, the coating of the flow path is non-binding with respect to the analyte, such as a metal-sensitive compound (e.g., a peptide). Consequently, the analyte, such as peptide compounds, does not bind to the coating of the flow path.

The alkylsilyl coating can be provided throughout the system from the tubing or fluid conduits 110 extending from the fluid manager system 105 all the way through to the detector 150. The coatings can also be applied to portions of the fluidic fluid path (e.g., at least a portion of the fluidic path). That is, one may choose to coat one or more components or portions of a component and not the entire fluidic path. For example, the internal portions of the column 125 and its frits 120 and end caps 115 can be coated whereas the remainder of the flow path can be left unmodified. Further, removable/replaceable components can be coated. For example, the vial or sinker 130 containing the sample reservoir can be coated as well as frits 120.

In one aspect, the flow path of the fluidic systems described herein is defined at least in part by an interior surface of tubing. In another aspect, the flow path of the fluidic systems described herein is defined at least in part by an interior surface of microfabricated fluid conduits. In another aspect, the flow path of the fluidic systems described herein is defined at least in part by an interior surface of a column. In another aspect, the flow path of the fluidic systems described herein is defined at least in part by passageways through a frit. In another aspect, the flow path of the fluidic systems described herein is defined at least in part by an interior surface of a sample injection needle. In another aspect, the flow path of the fluidic systems described herein extends from the interior surface of a sample injection needle throughout the interior surface of a column. In another aspect, the flow path extends from a sample reservoir container (e.g., sinker) disposed upstream of and in fluidic communication with the interior surface of a sample injection needle throughout the fluidic system to a connector/port to a detector. That is, all tubing, connectors, frits, membranes, sample reservoirs, and fluidic passageways along this fluidic path (wetted surfaces) are coated.

In some embodiments, only the wetted surfaces of the chromatographic column and the components located upstream of the chromatographic column are LBS, coated with the alkylsilyl coatings described herein, while wetted surfaces located downstream of the column are not coated. In other embodiments, all wetted surfaces are coated, including those surfaces downstream of the column. And in certain embodiments, wetted surfaces upstream of the column, through the column, and downstream of the column to the entrance of inlet to the detector are coated. The coating can be applied to the wetted surfaces via vapor deposition. Similarly, the “wetted surfaces” of labware or other fluid processing devices may benefit from alkylsilyl coatings described herein. The “wetted surfaces” of these devices not only include the fluidic flow path, but also elements that reside within the fluidic flow path. For example, frits and/or membranes within a solid phase extraction device come in contact with fluidic samples. As a result, not only the internal walls within a solid phase extraction device, but also any frits/membranes are included within the scope of “wetted surfaces.” All “wetted surfaces” or at least some portion of the “wetted surfaces” can be improved or tailored for a particular analysis or procedure by including one or more of the coatings described herein. The term “wetted surfaces” refers to all surfaces within a separation device (e.g., chromatography column, chromatography injection system, chromatography fluid handling system, frit, etc.). The term can also apply to surfaces within labware or other sample preparation devices (e.g., extraction devices) that come into contact with a fluid, especially a fluid containing an analyte of interest.

Further information regarding the coating and the deposition of coatings in accordance with the present technology is available in US 2019/0086371, which is hereby incorporated by reference.

In some examples, coating the flow path includes uniformly distributing the coating about the flow path, such that the walls defining the flow path are entirely coated. In some embodiments, uniformly distributing the coating can provide a uniform thickness of the coating about the flow path. In general, the coating uniformly covers the wetted surfaces such that there are no “bare” or uncoated spots.

Commercially available vapor deposition coatings can be used in the disclosed systems, devices, and methods, including but not limited to Dursan® and Dursox® (commercially available from SilcoTek Corporation, Bellefonte, Pa.).

Alkylsilyl coatings include bis(trichlorosilyl)ethane or bis(trimethoxysilyl)ethane (also known as C2) coatings. In some embodiments, the alkylsilyl coatings include two or more layers. For example, a first layer including C2 can be vapor deposited followed by a second layer of C10 material (n-decyltrichlorosilane). US Patent Publication No. US2019/0086371 (and in particular, Table 1) provides numerous examples of illustrative embodiments.

The coatings described above can be used to create LBS and can tailor a fluidic flow path of a chromatography system for the separation of a sample. The coatings can be vapor deposited. In general, the deposited coatings can be used to adjust the hydrophobicity of internal surfaces of the fluidic flow path that come into contact with a fluid (i.e. wetted surfaces or surfaces coming into contact with the mobile phase and/or sample/analyte). By coating wetted surfaces of one or more components of a flow path within a chromatography system, a user can tailor the wetted surfaces to provide a desired interaction (i.e., a lack of interaction) between the flow path and fluids therein (including any sample, such as a sample containing peptides, within the fluid).

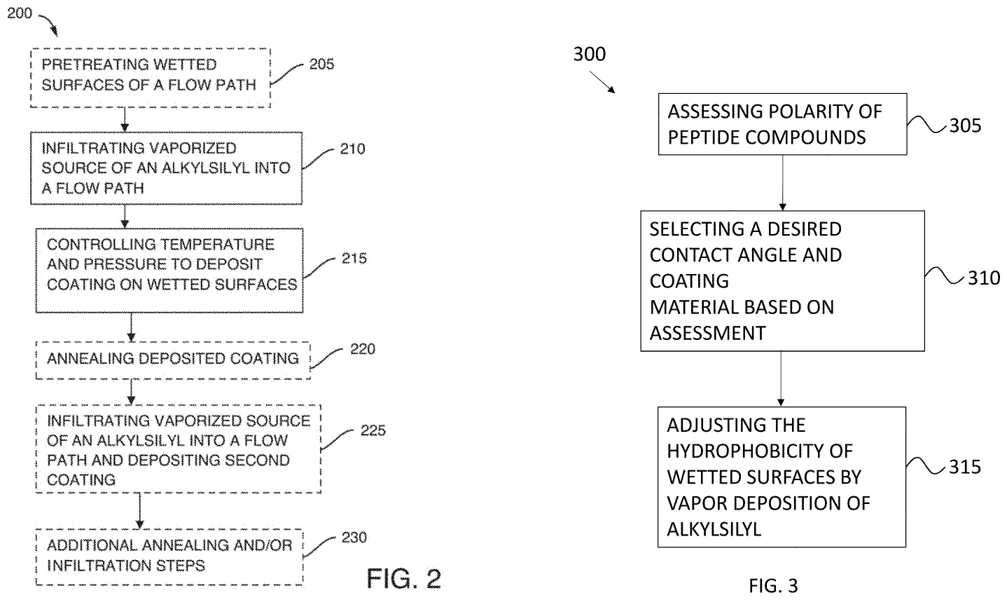

FIG. 2 is a flow chart illustrating method 200 for creating a LBS by tailoring a fluidic flow path for separation of a sample including peptide compounds. The method has certain steps which are optional as indicated by the dashed outline surrounding a particular step. Method 200 can start with a pretreatment step (205) for cleaning and/or preparing a flow path within a component for tailoring. Pretreatment step 205 can include cleaning the flow path with plasma, such as oxygen plasma. This pretreatment step is optional.

Next, an infiltration step (210) is initiated. A vaporized source of an alkylsilyl compound is infiltrated into the flow path. The vaporized source is free to travel throughout and along the internal surfaces of the flow path. Temperature and/or pressure is controlled during infiltration such that the vaporized source is allowed to permeate throughout the internal flow path and to deposit a coating from the vaporized source on the exposed surface (e.g., wetted surfaces) of the flow path as shown in step 215. Additional steps can be taken to further tailor the flow path. For example, after the coating is deposited, it can be heat treated or annealed (step 220) to create cross linking within the deposited coating and/or to adjust the contact angle or hydrophobicity of the coating. Additionally or alternatively, a second coating of alkylsilyl compound (having the same or different form) can be deposited by infiltrating a vaporized source into the flow path and depositing a second or additional layers in contact with the first deposited layer as shown in step 225. After the deposition of each coating layer, an annealing step can occur. Numerous infiltration and annealing steps can be provided to tailor the flow path accordingly (step 230).

FIG. 3 provides a flow chart illustrating a method (300) of creating a LBS by tailoring a fluidic flow path for separation of a sample including a analyte, such as peptide compounds. The method can be used to tailor a flow system for use in isolating, separating, and/or analyzing peptide compounds. In step 305, peptide compounds are assessed to determine polarity. Understanding the polarity will allow an operator to select (by either look up table or make a determination) a desired coating chemistry and, optionally, contact angle as shown in step 310.

In some embodiments, in addition to assessing the polarity of peptide compounds, the polarity of a stationary phase to be used to separate the peptide(s) (e.g., stationary phase to be included in at least a portion of the fluidic flow path) is also assessed. A chromatographic media (e.g., stationary phase) can be selected based on metal-sensitive compounds, e.g., peptide compounds, in the sample. Understanding the polarity of metal-sensitive compounds (e.g., peptide, acidic sample) and the stationary phase is used in certain embodiments by the operator to select the desired coating chemistry and contact angle in step 310. The components to be tailored can then be positioned within a chemical infiltration system with environmental control (e.g., pressure, atmosphere, temperature, etc.) and precursor materials are infiltrated into the flow path of the component to deposit one or more coatings along the wetted surfaces to adjust the hydrophobicity as shown in step 315. During any one of infiltration, deposition, and condition steps (e.g. annealing), coatings deposited from the infiltration system can be monitored and if necessary precursors and or depositing conditions can be adjusted if required allowing for fine tuning of coating properties.

### Methods for Preparing Samples

Prior to any comparisons of coated column/hardware performance versus uncoated column/hardware performance for peptide mapping and acidic samples, the following protocols were developed and used for sample preparation and analysis.

### Peptide Digest

For peptide mapping, a lyophilized NIST mAb digest was used (commercially available from Waters Corp., Milford, Mass., as Waters Corp. PN #186009126). Standards were reconstituted with 80 μL of 0.1% FA at a concentration of 0.5 mg/mL. Samples were then pooled, vortexed, and re-aliquoted in 200 μL aliquots into Eppendorf LoBind 0.5 μL tubes (available from Eppendorf, Hauppauge, N.Y.). Samples were stored at −80° C. prior to use. Upon use, samples were thawed, vortexed, and gently centrifuged prior to being placed in the autosampler.

As shown in Tables 1 and 2, the same gradient was used for both non-treated surfaces (CSH) and low binding surfaces (LBS).

TABLE 1
CSH peptide mapping (Gradient 1)

| Time (minutes) | Flow (mL/min) | % A | % B | Curve |
|---|---|---|---|---|
| 0 | 0.200 | 99 | 1 | Initial |
| 2 | 0.200 | 99 | 1 | 6 |
| 52 | 0.200 | 65 | 35 | 6 |
| 57 | 0.200 | 15 | 85 | 6 |
| 62 | 0.200 | 15 | 85 | 6 |
| 67 | 0.200 | 99 | 1 | 6 |
| 80 | 0.200 | 99 | 1 | 6 |

For peptide mapping, the system configuration including the mobile phase was as follows:

System Configuration (Using an ACQUITY™ System Commercially Available from Waters Technologies Corp., Milford, Mass.)

### Pump: ACQUITY™ Binary Solvent Manager

50 μL mixer

### Autosampler: ACQUITY™ AS-FTN

sample temperature=10° C.

Column manager: ACQUITY™ CM-A

column: CSH 2.1×100 mm, 1.7 μm

column temp=60° C.

Optical detector: ACQUITY™ TUV

FC—10 mm analytical, rate=10 Hz, λ=214 nm

Mass detector: ACQUITY™ QDa Mass Detector (high performance model)

rate=2 Hz, Cap.=1.5 kV, CV=10V, Probe=600° C.

### Mobile Phase

Mobile Phase A: Water, 0.1% formic acid (FA)
Mobile Phase B: MeCN, 0.1% formic acid (FA)

### Acidic Ladders

For acidic ladders, each synthetic peptide was manufactured from New England Peptide (Gardner, Mass.) and delivered as a lyophilized powder with ˜5 mg of sample in each vial. Using the absolute sample weight (as per manufacturer) each peptide stock solutions were prepared by reconstituting individual standards in water v/v 0.1% FA at a concentration of 1 mg/mL. Acidic ladders mixtures were then prepared of alternating standards 0E, 2E, and 4E for one mixture and 1E and 3E for the second mixture to provide equal UV intensity that were within the dynamic range of the assay. The final mixture for the 0E, 2E, 4E sample was: 0.012 mg/mL 0E, 0.008 mg/mL 2E, and 0.001 mg/mL 4E in 0.1% FA water. The final mixture for 1E and 3E was 0.004 mg/mL 1E and 0.004 mg/mL 3E.

TABLE 2
Acidic Ladder gradient (Gradient 2)

| Time (minutes) | Flow (mL/min) | % A | % B | Curve |
|---|---|---|---|---|
| 0 | 0.200 | 99 | 1 | Initial |
| 2 | 0.200 | 99 | 1 | 6 |
| 2.01 | 0.200 | 89 | 11 | 6 |
| 10 | 0.200 | 89 | 11 | 6 |
| 11 | 0.200 | 15 | 85 | 6 |
| 15 | 0.200 | 15 | 85 | 6 |
| 16 | 0.200 | 99 | 1 | 6 |
| 18 | 0.200 | 99 | 1 | 6 |

For peptide acidic ladders, the system configuration including the mobile phase was:

System Configuration (Using an ACQUITY™ System Commercially Available from Waters Corp., Milford, Mass.)

### Pump: ACQUITY™ Binary Solvent Manager

50 μL mixer

### Autosampler: ACQUITY™ AS-FTN

sample temp.=10° C.

Column manager: ACQUITY™ CM-A

column: CSH 2.1×100 mm, 1.7 μm

column temp=60° C.

Optical detector: ACQUITY™ TUV

FC—10 mm analytical, rate=10 Hz, λ=214 nm

Mass detector: ACQUITY™ QDa Mass Detector (high performance model)

rate=2 Hz, Cap.=1.5 kV, CV=10V, Probe=600° C.

### Mobile Phase

Mobile Phase A: Water, 0.1% formic acid
Mobile Phase B: MeCN, 0.1% formic acid

### Summary of Sample Types for Conventional Uncoated Columns Analysis

TABLE 3
Sample acquisition details

| | | | | | MS | | | |
|---|---|---|---|---|---|---|---|---|
| | | | | Avg. MW | scan | | Column | |
| Sample | type | identity | sequence | (g/mol) | type | m/z | type | Grad. |
| NIST | Peptide | — | — | — | TIC | 250- | CSH | 1 |
| mAb | | | | | | 1250 | | |
| trypsin | | | | | | | | |
| digest | | | | | | | | |
| NIST | Peptide | 1:T37 | GFYPSDIAVEW | 2535.38 | SIR | 849.20 | CSH | 1 |
| mAb | | | ESNGQPENNYK | | | | | |
| trypsin | | | (SEQ ID NO: 1) | | | | | |
| digest | | | | | | | | |
| NIST | Peptide | 1:T14 | VDNALQSGNSQ | 3191.97 | SIR | 713.00 | CSH | 1 |
| mAb | | | ESVTEQDSK | | | | | |
| trypsin | | | (SEQ ID NO: 3) | | | | | |
| digest | | | | | | | | |
| E-series | Peptide | — | — | — | TIC | 250- | CSH | 2 |
| acidic | | | | | | 1250 | | |
| ladder | | | | | | | | |
| E-series | Peptide | OE | VSNALQSGSSQS | 1969.04 | SIR | 985.0 | CSH | 2 |
| acidic | | | SVTSQSSK (SEQ | | | | | |
| ladder | | | ID NO: 4) | | | | | |
| E-series | Peptide | 1E | VENALQSGSSQ | 2011.07 | SIR | 1006.06 | CSH | 2 |
| acidic | | | SSVTSQSSK | | | | | |
| ladder | | | (SEQ ID NO: 5) | | | | | |
| E-series | Peptide | 2E | VENALQSGSSQ | 2053.11 | SIR | 1027.07 | CSH | 2 |
| acidic | | | ESVTSQSSK | | | | | |
| ladder | | | (SEQ ID NO: 6) | | | | | |
| E-series | Peptide | 3E | VENALQSGSSQ | 2095.15 | SIR | 1048.41 | CSH | 2 |
| acidic | | | ESVTEQSSK | | | | | |
| ladder | | | (SEQ ID NO: 7) | | | | | |
| E-series | Peptide | 4E | VENALQSGSSQ | 2137.18 | SIR | 1069.56 | CSH | 2 |
| acidic | | | ESVTEQESK | | | | | |
| ladder | | | (SEQ ID NO: 8) | | | | | |

Table 3 provides pertinent information regarding sample acquisition details with regards to sample structure, MW, MS acquisition type (full scan/SIR) and associated masses acquired for data analysis.

### Representative Chromatograms for Peptide Digest Sample in Conventional Uncoated Column

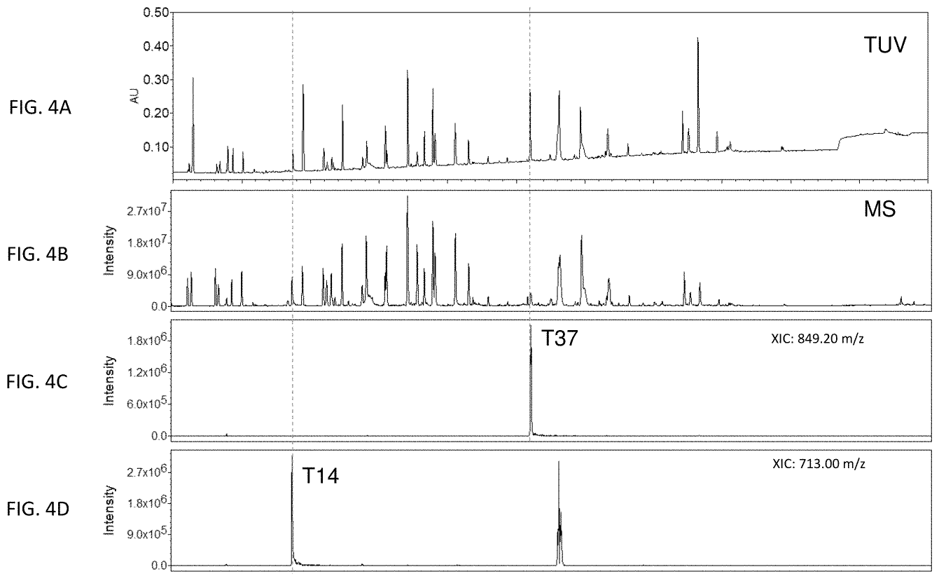

FIGS. 4A-4D are representative chromatograms for peptide mapping. FIG. 4A shows UV chromatogram of a peptide map of the NIST mAb digest using the method described in accordance with Table 1. FIG. 4B shows MS Total Ion Chromatogram (TIC) of the same peptide map acquired with the in-line QDa mass detector. FIG. 4C shows Extracted Ion Chromatogram (XIC) (849.20 m/z) of the T37 “acidic” peptide from the peptide map to indicate approximate elution region and profile. FIG. 4D shows XIC (713.00 m/z) of the T14 “acidic” peptide from the peptide map to indicate approximate elution region and profile.

### Representative Chromatograms for Acidic Ladder Samples in Conventional Uncoated Column

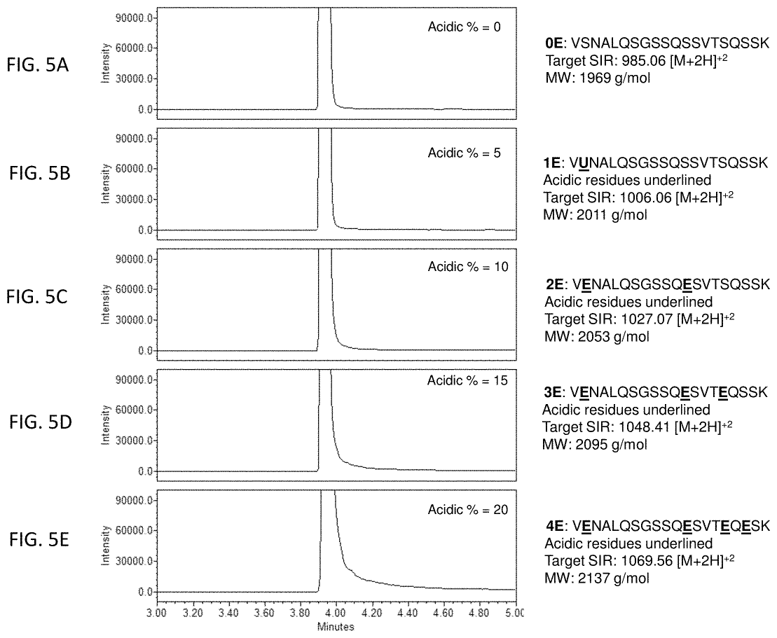

FIGS. 5A-5E are representative acidic ladder chromatograms for glutamic acid “E” series. Peptides were synthetically manufactured and chromatographically separated under step gradient conditions shown in Table 2 with the following sequences.

FIG. 5A included VSNALQSGSSQSSVTSQSSK (SEQ ID NO: 4)=0E with acidic % equal to 0, target selected ion recording (SIR) of 985.06 [M+2H]⁺² and molecular weight (MW) of 1969 g/mol.

FIG. 5B included VENALQSGSSQSSVTSQSSK (SEQ ID NO: 5)=1E with acidic % equal to 5, target SIR of 1006.06 [M+2H]⁺² and MW of 2011 g/mol.

FIG. 5C included VENALQSGSSQESVTSQSSK (SEQ ID NO: 6)=2E with acidic % equal to 10, target SIR and of 1027.07 [M+2H]⁺² and MW of 2053 g/mol.

FIG. 5D included VENALQSGSSQESVTEQSSK (SEQ ID NO: 7)=3E with acidic % equal to 15, target SIR of 1048.41 [M+2H]⁺² and MW of 2095 g/mol.

FIG. 5E included VENALQSGSSQESVTEQESK (SEQ ID NO: 8)=4E with acidic % equal to 20, target SIR of 1069.56 [M+2H]⁺² and MW of 2137 g/mol. FIGS. 5A-5E were used to evaluate increasing acidic character impact on tailing. Data was acquired using the QDa in selected-ion-recording (SIR) mode with the [M+2H]⁺² charge state being acquired for each peptide.

### Calculation of Tailing Factor

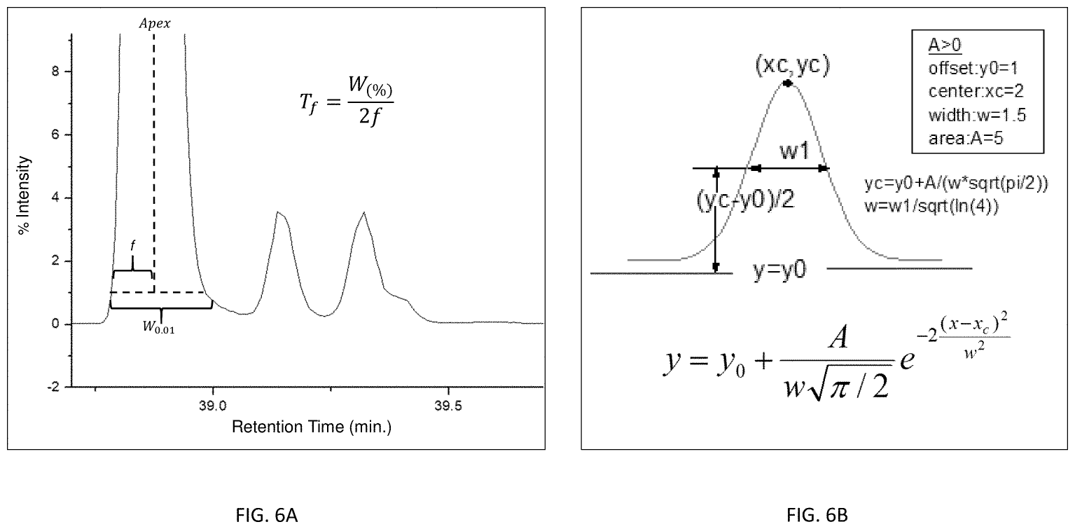

FIGS. 6A and 6B display how chromatographic performance was evaluated for the samples separated in the uncoated conventional columns described above. This same calculation is used in the comparative examples below including coated hardware of the present technology. FIG. 6A shows tailing factor, T_{f}, defined as the peak width in minutes at an assigned peak height based on relative peak intensity (W_{%}), divided by 2 times a fraction of the peak width defined as the first half of the peak as determined by the peak apex. FIG. 6B shows the peak width, W, based on a Gaussian fit function where W equals 2σ.

### Comparative Examples

The following conditions were used to evaluate LBS-based methods for peptide mapping workflow. A single metal-based LC system was configured where the sample flow path components were used (a) as is (unmodified) or (b) substituted with instrument parts and/or columns containing inert surfaces (LBS) for comparison studies. A commercially available lyophilized NIST mAb digest was used for all experiments. Samples were separated on a C18 reversed phase chemistry (CSH C18 columns) using a 50-min gradient at 0.68% B/min with 0.1% formic acid as a mobile phase additive. When the system is in an unmodified configuration, no coating/LBS surfaces are incorporated (non-coated hardware). When the system is in a substituted configuration, coatings/LBS are incorporated into the system along the flow path (LBS-coated hardware). In particular, the following examples illustrate improved tailing results (i.e., reduced peak tailing), improved reproducibility, and improved selectivity for the coated hardware of the present technology.

### Reduced Tailing

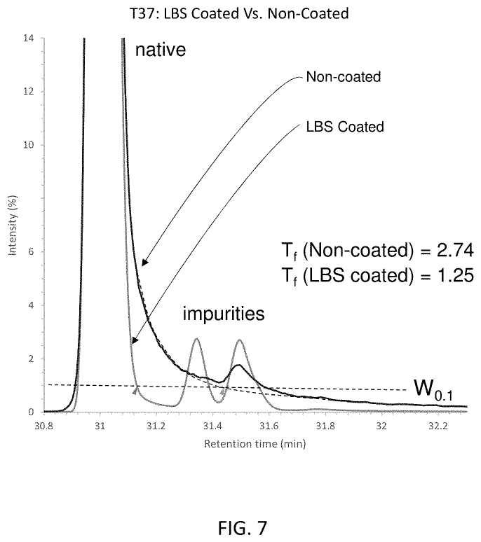

FIG. 7 displays the tailing factor that was evaluated for the T37: GFYPSDIAVEWESNGQPENNYK acidic peptide (SEQ ID NO: 1) (T37-PENNYK (SEQ ID NO: 2)) using the selected ion recording (SIR) function of the QDa with a m/z of 849.20 being acquired for evaluation. This peptide contains 3 glutamic acids and 1 aspartic acid residue, which represent 18% of the peptide sequence. T37 is known to be susceptible to post translation modifications such as deamidation where in an amide functional group in the side chain of the amino acid is removed or converted to another functional group. This is often asparagine (N) being converted to aspartic or isoaspartic acid impurities.

As shown in the figure, peptides containing acidic residues such as T37 exhibit a high degree of tailing. In this example, the non-coated or untreated metal flow path resulted in a tailing factor value of 2.74 for the T37 peptide (top line as indicated in FIG. 7). Only 1 of the deamidated impurities is partially resolved from the native peak due to excessive tailing of the native peak and potential tailing of the impurities themselves. Tailing of T37 was reduced by 54% to a value of 1.25 when the same sample was separated using the same method on the same system configured with Low Binding Surface (LBS) parts comprised of the injection needle, need port assembly, active pre-heater, and column hardware bearing a C2 coating (bottom line as indicated in FIG. 7). The reduced tailing of the native peak facilitated the chromatographic separation of the both deamidated impurities which were approximately baseline resolved from the native peak. The triangles of the non-coated and LBS coated lines at the dashed line at W_{0.1} represent where the trailing edge of the peak was approximated for determination of tailing factor values for the associated chromatographic trace. The non-coated time offset was −0.78 minutes.

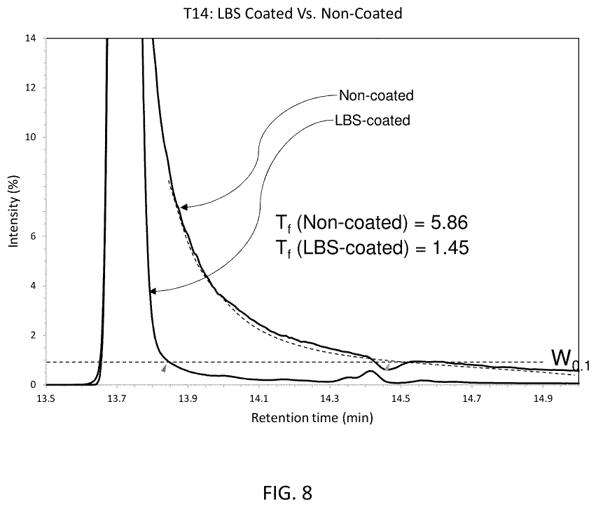

FIG. 8 displays the T14 tailing factor that was evaluated for the T14: VDNALQSGNSQESVTEQDSK acidic peptide (SEQ ID NO: 3) using the selected ion recording (SIR) function of the QDa with an m/z of 713.00 being acquired for evaluation. This peptide contains 2 glutamic acids and 2 aspartic acid residues which represent 20% of the peptide sequence. As shown in the figure, peptides containing acidic residues such as T14 exhibit a high degree of tailing. In this example the non-coated or untreated metal flow path resulted in a tailing factor value of 5.86 for the T14 peptide (top line). Tailing of T14 was reduced by 75% to a value of 1.45 when the same sample was separated using the same method on the same system configured with Low Binding Surface (LBS) parts comprised of the injection needle, need port assembly, active pre-heater, and column hardware bearing a C2 coating (top line). The triangles of the non-coated and LBS coated lines at the dashed line at W_{0.1} represent where the trailing edge of the peak was approximated for determination of tailing factor values for the associated chromatographic trace. The non-coated time offset was −0.93 min.

For FIGS. 7 and 8, new peaks can be attributed to reduced tailing. In FIG. 7, the first eluting impurity peak at 31.3 in the non-coated data would not likely be integrated by software since the tailing peak is dominating the absorbance in that area. In FIG. 8, the suppression event in the MS response at 14.45 minutes of the non-coated trace is most likely due to the peaks observed in the coated results at the same time. LBS parts do not alter samples in a way that would be viewed as degradation or introducing new chromatographic artifacts resulting in peak loss or diminished recovery of sample.

### Reproducibility

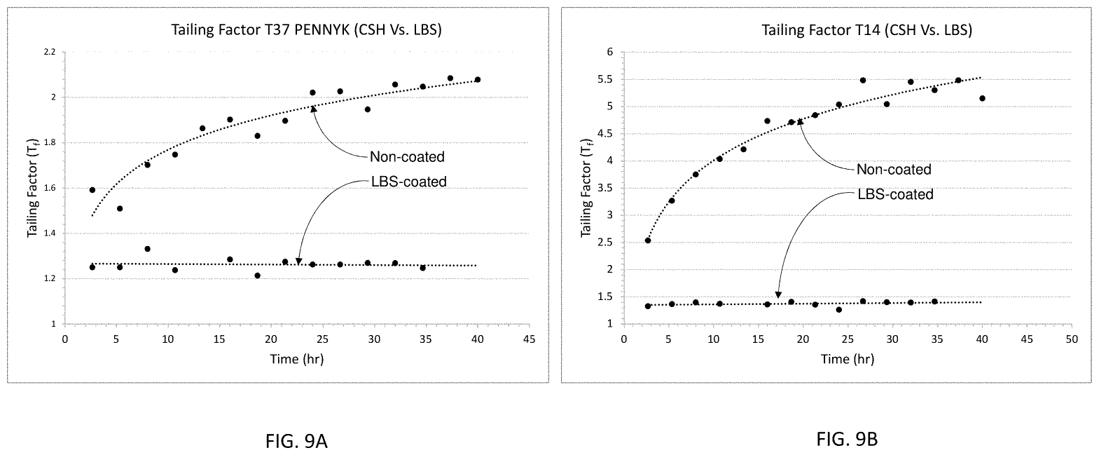

FIGS. 9A and 9B show the reproducibility of LBS surfaces. Tailing factor was plotted over time for acidic peptides T37 and T14 for both non-coated and LBS coated surfaces. Briefly, the liquid chromatography (LC) system was washed with phosphoric acid and rinsed until the pH was measured to be approximately seven, pH=7. A new non-coated column was placed in the system and 15 injections of samples intercalated with water blanks were performed using the gradient shown in Table 1. The process was then repeated with LBS hardware and column being placed in-line post phosphoric acid wash. As shown in the data for non-coated surfaces, tailing factor was observed to increase from the initial injection for both T37 and T14 acidic peptides when using non-coated parts and column. Both peptides appear to be approaching a saturation limit or “leveling-off” for tailing. This may be indicative of active sites being occupied over time by adsorbed analyte where a “conditioned” state is reached in the un-coated hardware configuration. Average tailing factor was determined to be 2.04 (Relative Standard Deviation (RSD)=8.87%) and 4.60 (RSD=19.07%) for T37 and T14, respectively.

In contrast, the tailing factor was significantly stable from initial injection onward for both T37 and T14 peptides when using LBS coated hardware and columns. Mean tailing factor was calculated to be 1.26 (RSD=1.01%) and 1.40 (RSD=0.63%) for T37 and T14, respectively. This data further supports the notion that active coatings can be used to mitigate secondary interactions with metal surfaces.

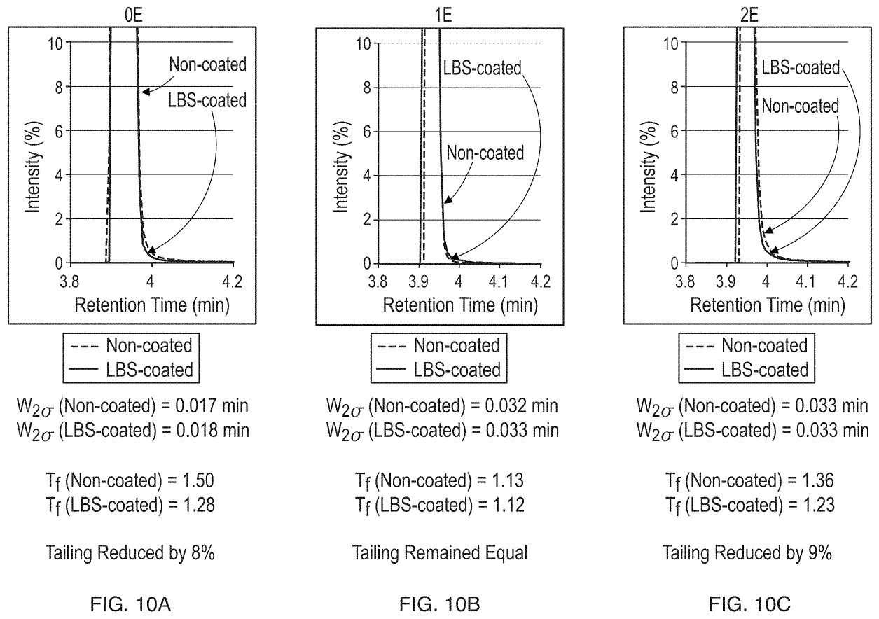

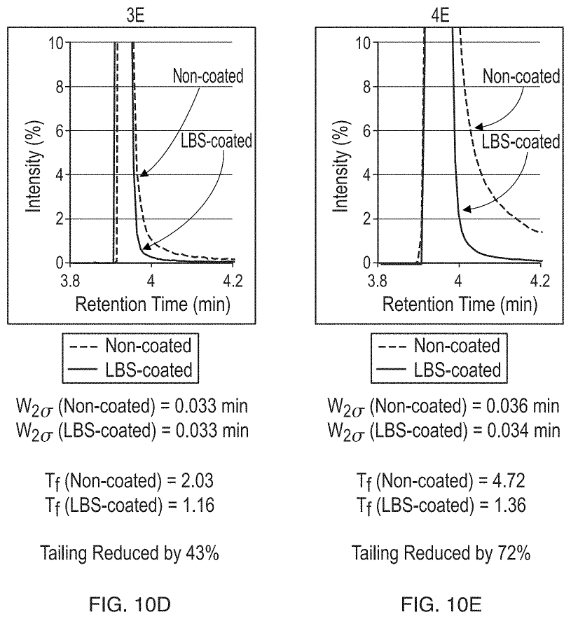

FIGS. 10A-10E show the acidic ladder, E-series, for non-coated and LBS coated surfaces. In FIGS. 10A-10E, the tailing factor was experimentally determined for a series of synthesized peptide sequences with increasing acidic property using a system configured with non-coated hardware and LBS coated hardware. Briefly, sequences

(SEQ ID NO: 4)
VSNALQSGSSQSSVTSQSSK = 0E (FIG. 10A),
(SEQ ID NO: 5)
VENALQSGSSQSSVTSQSSK = 1E (FIG. 10B),
(SEQ ID NO: 6)
VENALQSGSSQESVTSQSSK = 2E (FIG. 10C),
(SEQ ID NO: 7)
VENALQSGSSQESVTEQSSK = 3E (FIG. 10D),
(SEQ ID NO: 8)
VENALQSGSSQESVTEQESK = 4E (FIG. 10E),

were synthesized replacing targeted serine residues with glutamic acid to increase the acidic character of the peptide.

Using the step gradient conditions in Table 2, the acidic ladders were separated on a system containing either non-coated or LBS coated hardware and column. Prior to running the acidic ladders, columns for both non-coated and LBS coated evaluations were conditioned with 15 injections of a peptide digest of the NIST mAb standard as described in Tables 1 and 2 with water blanks run in between each standard run. Following conditioning, 15 injections of the acidic ladder mixtures were performed. As shown in FIGS. 10A-10E, the tailing factor generally increased with increasing glutamic acid content on the non-coated system with the 4E sequence exhibiting the highest amount of tailing with a tailing factor of 4.72.

In contrast, tailing was reduced up to 72% for the 4E sequence when separated on the LBS-coated hardware as shown in FIG. 10E. Furthermore, tailing was observed to be reduced in 0E, 1E, 2E, and 3E synthetic sequences.

Upon closer inspection it was also observed that LBS coated hardware does not appear to adversely affect chromatography performance as shown in the comparable peaks widths across the acidic ladder series. Specifically, the synthetic peptides with lower acidic character (0E<1E<2E<3E<4E) do not exhibit a hydrophobic affinity towards the LBS coating.

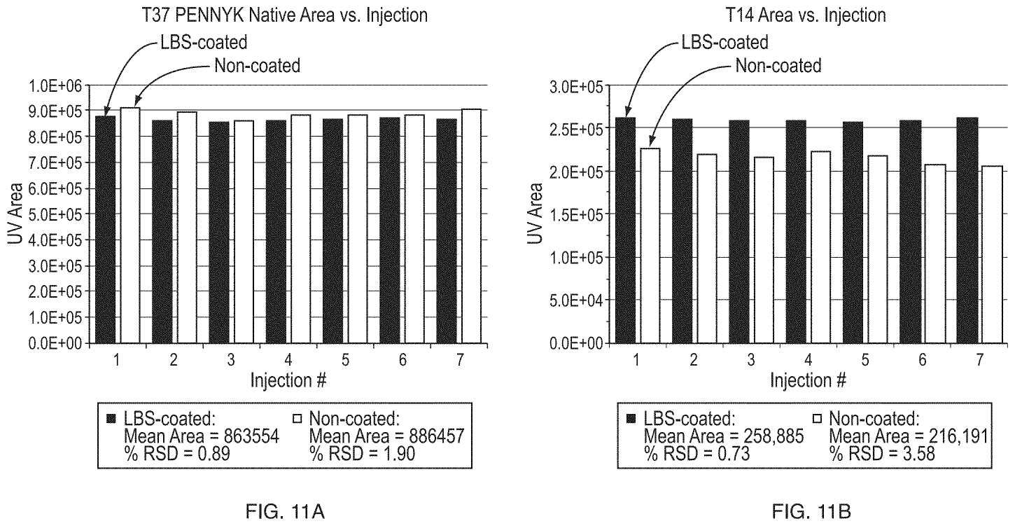

FIGS. 11A and 11B display peptide recovery and conditioning for non-coated and LBS coated surfaces. Peptide recovery was evaluated for both the T37 and T14 peptides from the NIST mAb digest (commercially available from Waters Corp., Milford, Mass., as Waters Corp. PN #186009126). Briefly, the LC system was washed with phosphoric acid and rinsed until the pH was measured to be approximately seven, pH=7. A new non-coated column was placed in the system and 15 injections of samples intercalated with water blanks were performed using the gradient shown in Table 1. The process was then repeated with LBS hardware and column being placed in-line post phosphoric acid wash. Using UV data, the area for the main native peak of peptide T37 and T14 were plotted for the first 7 injections. As shown in FIG. 11A, recovery of the T37 native peak was comparable between the non-coated and LBS-coated configurations with a mean area of 886,000 and 864,000 calculated for the non-coated and LBS coated hardware, respectively. LBS-coated hardware did show increased chromatographic stability from the initial injection with a % RSD=0.89 representing a 53% reduction in variability when compared to the non-coated hardware which had a % RSD=1.90.

As shown in FIG. 11B, recovery of the T14 native peak was markedly higher in the LBS-coated hardware in comparison to the non-coated hardware with a 20% improvement in peptide recovery based on a mean area of 259,000 and 216,000 for the LBS coated hardware and non-coated hardware, respectively. Furthermore, the LBS-coated hardware was observed to increase chromatographic stability of the T14 peptide from the initial injection with a % RSD=0.73 representing an 80% reduction in variability when compared to the non-coated hardware which had a % RSD=3.58.

### Selectivity

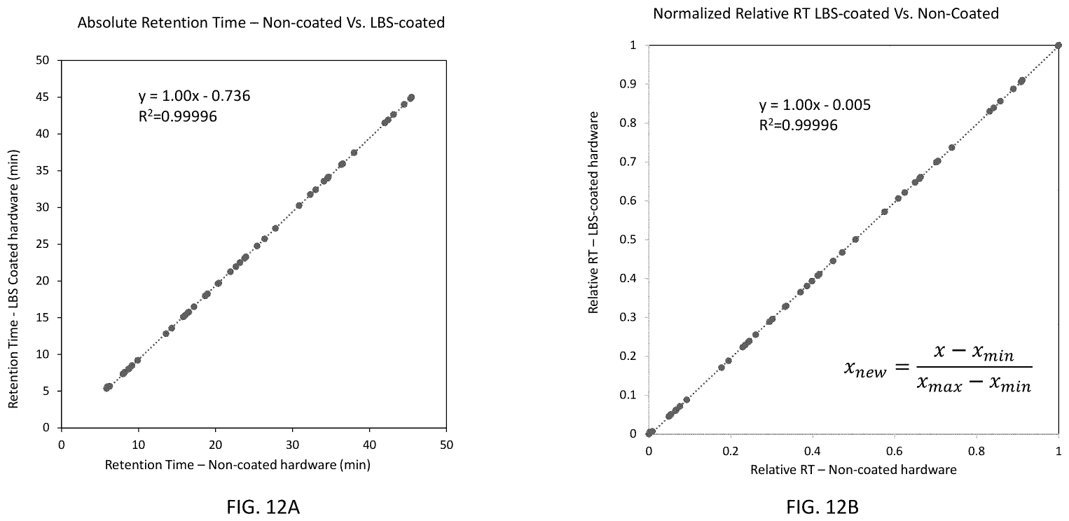

FIGS. 12A and 12B show the selectivity comparison of LBS-coated versus non-coated surfaces. Chromatographic selectivity was evaluated for all major peptides from the NIST mAb digest (commercially available from Waters Corp., Milford, Mass., as Waters Corp. PN #186009126) eluted using the gradient from Table 1 for both non-coated and coated hardware. Briefly, the LC system was washed with phosphoric acid and rinsed until the pH was measured to be approximately seven, pH=7. A new non-coated column was placed in the system and 15 injections of samples intercalated with water blanks were performed using the gradient shown in Table 1. The process was then repeated with LBS hardware and column being placed in-line post phosphoric acid wash. Using UV data from the 10^{th} injection of the injection series from both non-coated and LBS-coated hardware, peaks with a minimum S/N ratio of 3 or more were integrated and plotted against each other as a function of retention time. Normalized relative retention time was also evaluated for both non-coated and LBS-coated hardware by plotting the ratio of the normalized RT using the last eluting peak as the reference peak. As shown in FIG. 12A, the orthogonal comparison indicated good RT agreement between the non-coated and LBS-coated hardware runs with the fitted data exhibiting a slope value of 1.00 with an R²=0.99996. An overall systematic retention time shift was observed as indicated by the y-intercept value of −0.736. This may be due to minor variability in the flow path tubing length used in the different configurations. As shown in FIG. 12B, after normalization, the non-coated and LBS-coated configurations exhibited almost identical retention time profiles with the fitted data exhibiting a slope value of 1.00 and y-intercept value of −0.005, indicating column selectivity was negligibly impacted in the LBS-coated hardware.

### No Adverse Performance Observed

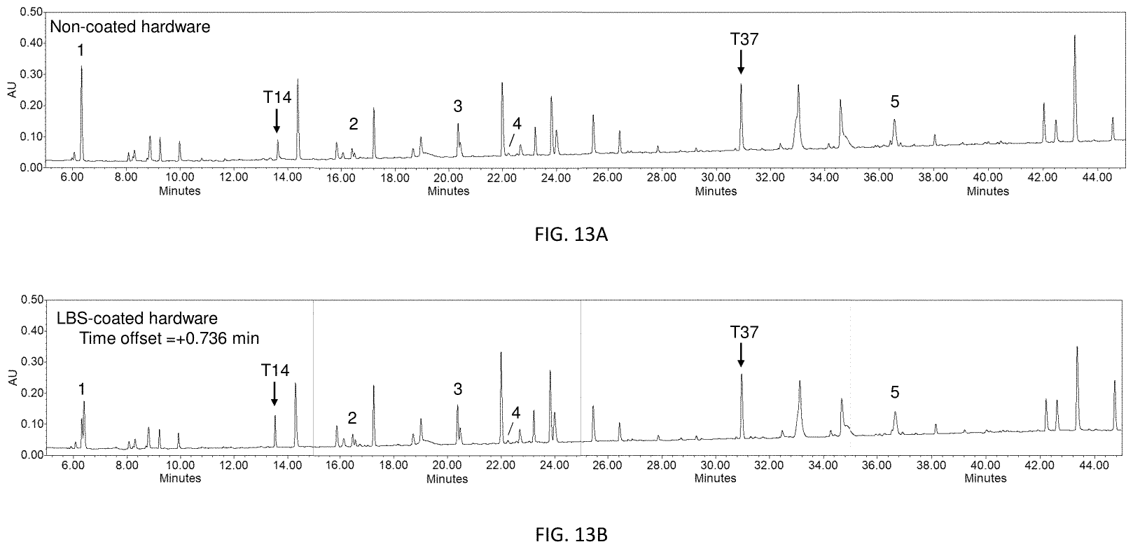

FIGS. 13A and 13B show a peptide profile comparison. Peptide profiles were evaluated for all major peptides from the NIST mAb digest (commercially available from Waters Corp., Milford, Mass., as Waters Corp. PN #186009126) eluted using the gradient from Table 1 for both FIG. 13A (non-coated hardware) and FIG. 13B (LBS-coated hardware). Briefly, the LC system was washed with phosphoric acid and rinsed until the pH was measured to be approximately seven, pH=7. A new non-coated column was placed in the system and 15 injections of samples intercalated with water blanks were performed using the gradient shown in Table 1. The process was then repeated with LBS hardware and column being placed in-line post phosphoric acid wash. Using UV data from the 8^{th} injection of the injection series from both non-coated and LBS-coated hardware, peptide map profiles were time aligned for comparison. As previously documented (FIGS. 7 and 8), the LBS-coated material did show quantitative improvement in peak tailing for peptides T14 and T37 as indicated in FIGS. 13A and 13B. From a qualitative perspective, further inspection of the peptide profiles indicate chromatographic performance improved for non-targeted peaks as noted in peaks 1-4. The apparent loss of resolution in peak 5 with the earlier eluting neighboring peak may be attributed to the minor differences in tubing length discussed in FIGS. 12A and 12B as gradient compositional differences could impact closely eluting critical pairs such as these. Holistically speaking though, no new peaks or absence of peaks were observed when comparing non-coated hardware results with LBS-coated hardware results, indicating LBS-coated hardware does not adversely impact chromatographic performance or recovery of peptides in RPLC-based separations of peptides using conditions outlined in Tables 1 and 2.

### Further Evaluation Including MS/MS

To obtain additional data and evaluation of the present technology, comparisons studies using non-coated and coated columns were conducted on systems using more sensitive MS detectors (i.e., detector with wider m/z range and ability to run in MSE mode). The experimental set up for this further evaluation is as follows:

A lyophilized NIST mAb digest was used (commercially available from Waters Corp., Milford, Mass., as Waters Corp. PN #186009126). Standards were dissolved in 200 μL of LC-MS grade water containing 0.1% FA at a concentration of 0.2 μg/μL. A 5 μL injection was performed for every LC-MS run loading 1.0 μg on column. This loading amount is recommended with 0.1 FA based reverse phase solvent systems.

The gradient applied is as shown in Table 4. The same gradient was used for both non-treated surfaces (CSH) and low binding surfaces (LBS).

TABLE 4
CSH peptide mapping (Gradient 1)

| Time (minutes) | Flow (mL/min) | % A | % B |
|---|---|---|---|
| 0 | 0.200 | 99 | 1 |
| 2 | 0.200 | 99 | 1 |
| 52 | 0.200 | 65 | 35 |
| 57 | 0.200 | 15 | 85 |
| 62 | 0.200 | 15 | 85 |
| 67 | 0.200 | 99 | 1 |
| 80 | 0.200 | 99 | 1 |

For peptide mapping, the system configuration including the mobile phase was as follows:

System Configuration (Using an ACQUITY™ System Commercially Available from Waters Corp., Milford, Mass.)

### Pump: ACQUITY™ Binary Solvent Manager

50 μL mixer

### Autosampler: ACQUITY™ AS-FTN

sample temperature=10° C.

Column manager: ACQUITY™ CM-A

column: CSH 2.1×100 mm, 1.7 μm

column temp=60° C.

Mass detector: ACQUITY™ RDa Mass Detector; (BioAccord System available from Waters Technology Corporation, Milford, Mass.)

### Ionization Mode: ESI Positive

Acquisition mode: Full MS scans with CID fragmentation (MS with fragmentation mode)
Acquisition range: m/z 50-2000

### Capillary Voltage 1.2 kV

Collision Energy 60-120 V (low-high energy ramping)

### Cone Voltage 20 V

Desolvation temperature 350 C
Intelligent data capture: on

### Mobile Phase

Mobile Phase A: Water, 0.1% formic acid (FA)
Mobile Phase B: MeCN, 0.1% formic acid (FA)

### Reduced Tailing

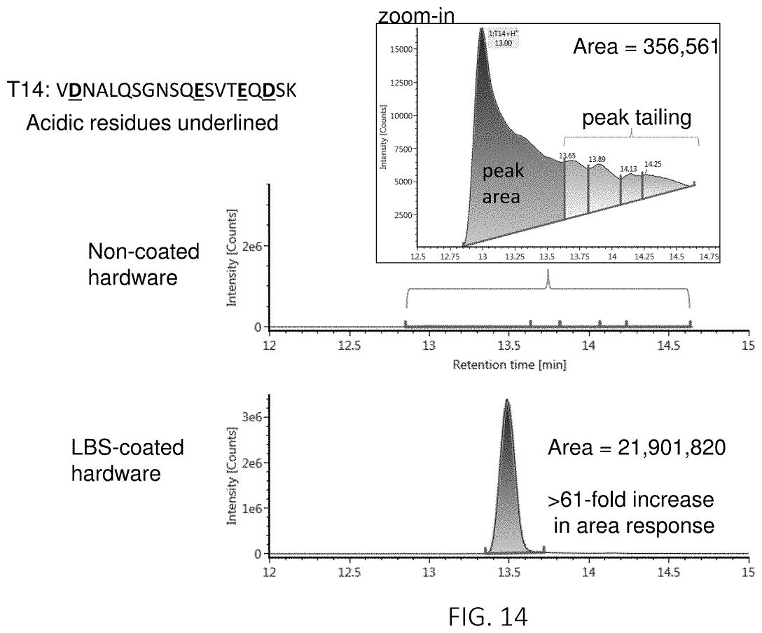

FIG. 14 displays the improvement in peak tailing for coated hardware. FIG. 14 provides an extracted ion chromatogram (XIC) of NISTmAb tryptic peptide T14 (VDNAKQSGNSQESVTEQDSK (SEQ ID NO: 9)) acquired on the BioAccord system using ACQUITY™ peptide CSH C18 (uncoated, conventional column) and a coated CSH C18 column (all columns and systems available from Waters Technologies Corporation, Milford, Mass.). The coated CSH C18 column was coated to provide low binding surfaces (LBS). For this example, the particular coating applied was a C2 coating, described in US Patent Publication 2019/0086371 (and incorporated by reference in its entirety). The XIC of CSH C18 peptide peak shows extensive peak tailing (top XIC of FIG. 14). The UNIFI peptide mapping method was used for peak area calculation of the MS response. Due to peak tailing the UNIFI workflow method was unable to correctly integrate CSH C18 (uncoated hardware), T14 XIC resulting in a skewed peak area measurement for the peptide. The blue area (identified as peak area) shows peak area integrated and used in MS response measurements and the yellow (identified as peak tailing) shows the areas of the peak integrated but not used in the measurement. In contrast the same peptide showed a 61-fold increase in area response with negligible tailing observed when using the coated hardware (i.e., coated to provide LBS). The differences in area observed are attributed to adsorptive losses of the acidic peptide to metal surfaces.

### MS Response

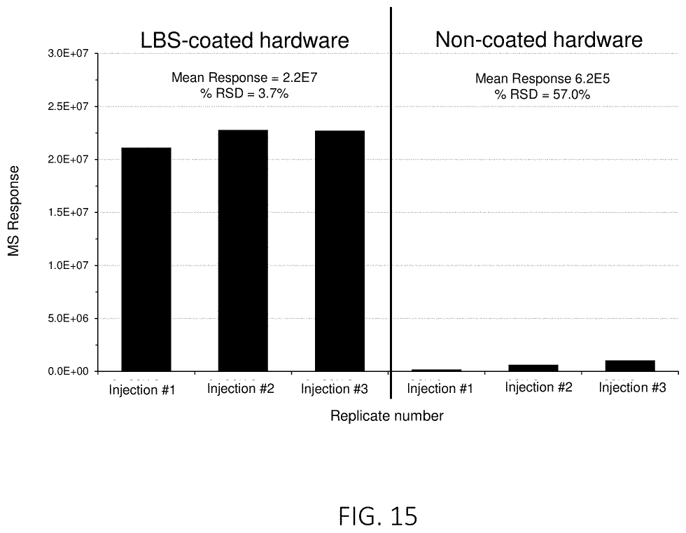

FIG. 15 shows the MS response for T14 (VDNALQSGNSQESVTEQDSK (SEQ ID NO: 3)) peptide monitored using UNIFI peptide mapping workflow reported for three replicate LC-MS injections performed on coated CSH C18 and CSH C18 (uncoated) columns. The MS response reported for T14 using CSH C18 is lower than the coated CSH C18 column. Further the coated CSH C18 column resulted in consistent MS responses (% RSD 3.7%) for T14 peptide compared to CSH C18 (uncoated) column (% RSD 57%).

Without wishing to be bound by theory, it is believed that the coated columns (i.e., coated hardware of the present technology) decrease peak tailing of this peptide peak resulting in a peak area accurately integrated by UNIFI peptide mapping workflow method in MS response calculations.

### Fragmentation Data

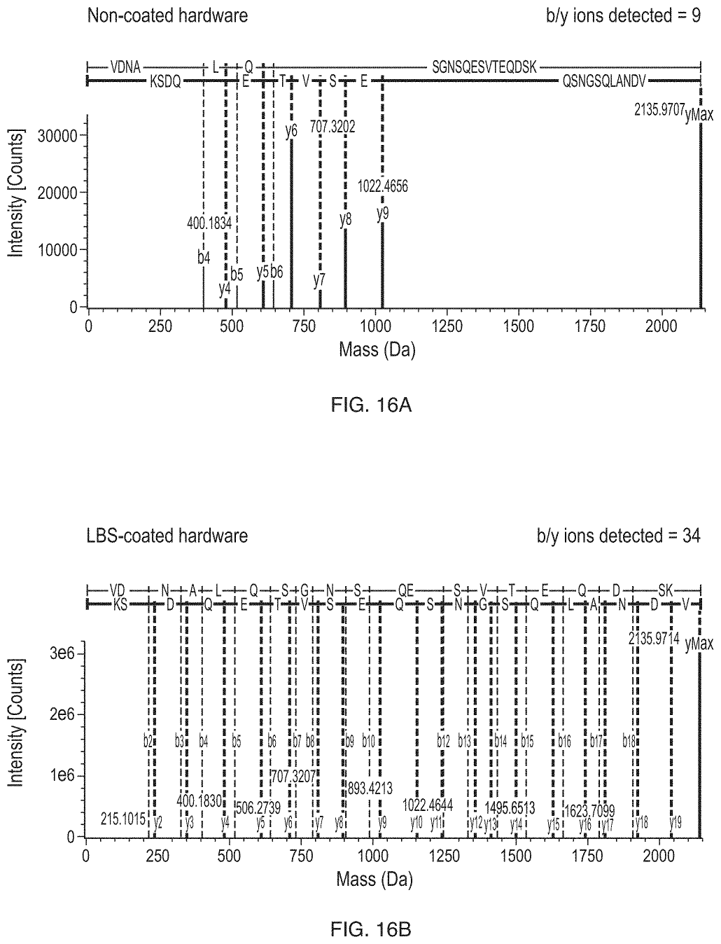

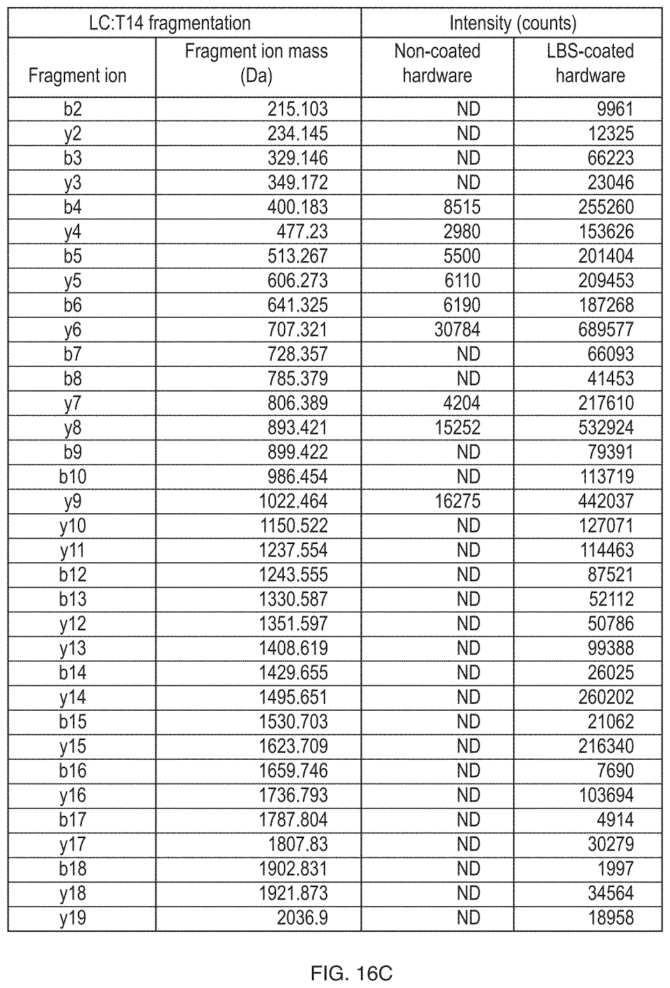

FIG. 16 illustrates the enhanced results and capabilities of utilizing the present technology. FIG. 16 is annotated fragmentation spectra for T14 NISTmAb tryptic peptide (VDNALQSGNSQESVTEQDSK (SEQ ID NO: 3)) generated by collision induced dissociation (CID). The blue (identified with b) and red (identified with y) lines show b and y fragment ions of the peptide backbone of T14. The fragment ion matching was performed during UNIFI peptide mapping data processing using the workflow method. The coated CSH C18 column (lower spectra, FIG. 16B) shows higher number of b and y ions (34 ions) in the annotated fragmentation spectrum compared to the uncoated CSH C18 (FIG. 16A top spectra, the maximum fragment ions observed was only 9 b/y ions). Majority of the ions observed with the coated CSH C18 column (column coated to provide LBS) was not seen in the uncoated CSH C18 data. This is due to increased MS intensity observed with the coated column for T14 peptide that subsequently generate high intensity fragment ions following CID. This phenomenon attributes to the missing b/y ions in the uncoated CSH C18 data which could be below limit of detection of the instrument. The fragmentation data is often used to confirm a peptide's sequence and high number of fragment ions results in high confidence peptide identifications.

### Improved Peptide Mapping

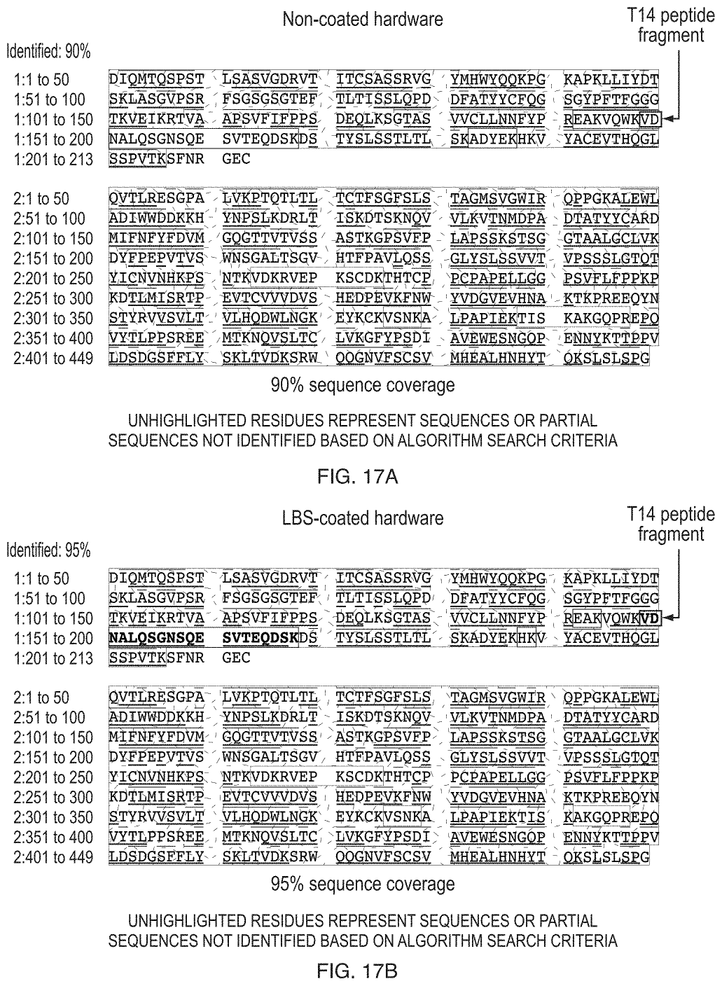

With less analyte loss (by mitigating metal-ion adsorption of sensitive analytes) comes improved recovery and sensitivity. As a result, increased sequence coverage is possible, leading to an improved mapping ability. The data shown in FIG. 17A and FIG. 17B illustrates the improvements achieved with LBS coated hardware of the present technology over uncoated conventional hardware. Specifically, the data shows protein sequence coverage observed for NISTmAb digest standard with uncoated CSH C18 and coated CSH C18 columns (i.e., CSH C18 column coated with C2 along wetted surfaces to provide LBS). The sequence coverage of the protein observed with the two column are: uncoated CSH C18 at 90% and coated CSH C18 at 95%. A filtering criteria is often utilized in UNIFI based peptide mapping analysis to validate peptide sequences identified in the analysis. The criteria used in the analysis are: no insource fragment ions including ammonia or water losses, mass accuracy between −10 ppm and 10 ppm, the minimum number of b/y fragment ions for peptide is greater than or equal to 5. The difference in sequence coverage is mainly due to lower intensity observed for T14 peptide with uncoated CSH C18 column resulting in insufficient number of b/y fragment ions (≥5 ions required) than the coated column (i.e., CSH C18 column coated to provide LBS). The remaining missed identifications are due to short peptide sequences that did not have sufficient fragmentation data (contained<5 b/y ions per peptide) to be included in sequence coverage measurements or were not retained by the column due to low hydrophobicity to be sufficiently adsorbed to the stationary phase.

### Reduction in Metal Adducts

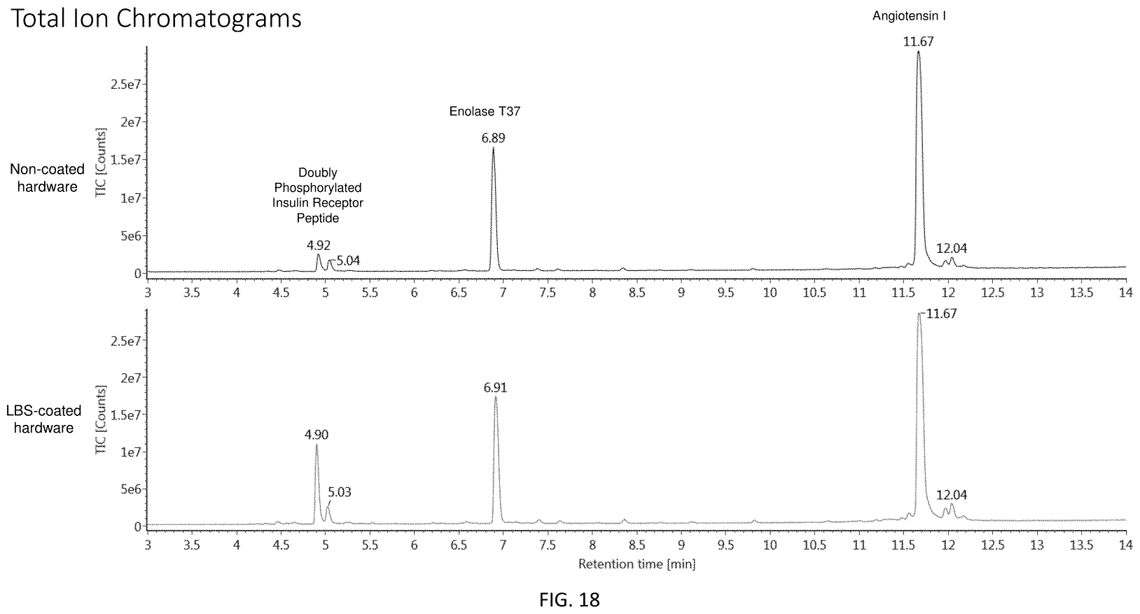

Data in FIGS. 18-24 is provided to demonstrate the reduction of metal adducts in ESI-MS assays when using coated hardware in accordance with the present technology. Three sample types are evaluated in this example: a doubly phosphorylated insulin receptor peptide, Enolase T37, and Angiotensin I. The data demonstrates reduction in metal adducts, specifically a reduction in iron adducts from either mobile phase impurities or metal components such as stainless steel. By extension this benefit could be extrapolated to other trace metal impurities found in either the mobile phase or potentially those that could be leaching out of metal components in the instrument hardware. This could include metal adducts such as Ni for nickel cobalt alloys or titanium (Ti) for hardware components manufactured or containing titanium.

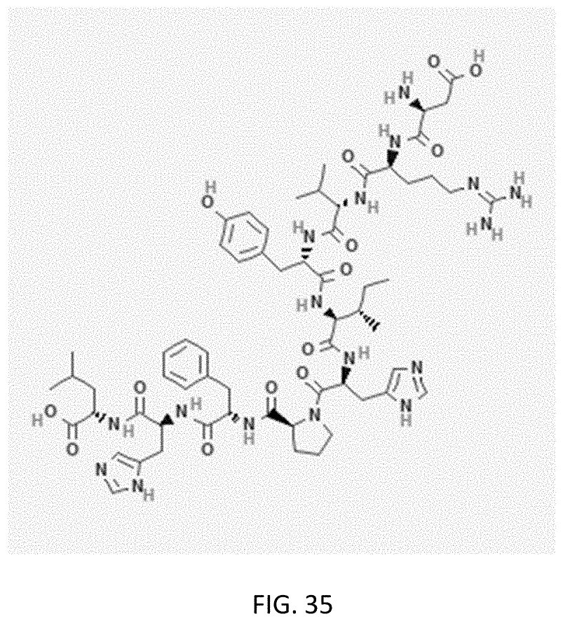

The first sample analyzed was doubly phosphorylated insulin receptor peptide. It is a peptide with a with a sequence of: TRDI(pY)ETD(pY)YRK (SEQ ID NO: 10). It has a molecular weight of 1782.6 Da. A lyophilized pellet of doubly phosphorylated insulin receptor was reconstituted in 0.1% formic acid in water to yield a 500 pmol/μL concentration sample for use with conditioning steps. This sample was further diluted to 60 pmol/μL to make the three peptide mixture. The second sample analyzed was Enolase T37. The second sample is a synthetic peptide derived from Enolase having the sequence YPIVSIEDPFAEDDWEAWSHFFK (SEQ ID NO: 11). It is an acidic peptide exhibiting a pI of 3.97 and molecular weight of 2829.1 Da. A lyophilized pellet of Enolase T37 will be reconstituted in 0.1% TFA in water with 10% DMSO to yield a final concentration of 353 pmol/μL This sample will be further diluted to 60 pmol/μL to make the three peptide mixture and 12.5 pmol/μL for the recovery sample. The third sample was Angiotensin I. FIG. 35 displays the structure of Angiotensin I. Angiotensin I is a peptide with a sequence of: Asp-Arg-Val-Tyr-Ile-His-Pro-Phe-His-Leu (SEQ ID NO: 12). It has a molecular weight of 1296.48 Da. A lyophilized pellet of Angiotensin I will be reconstituted in 0.1% TFA in water with 10% DMSO to give a concentration of 771 pmol/μL. This sample will be further diluted to 60 pmol/μL to make the three peptide mixture.

To obtain the data presented in FIGS. 18-24, equal volumes of 60 pmol/μL Angiotensin I, Enolase T37, and doubly phosphorylated insulin receptor peptide was mixed to prepare a 1:1:1 equimolar mixture having a final concentration for each peptide that is approximately 20 pmol/μL.

The following experimental parameters were used:

LC System Parameters

| | |
|---|---|
| System: | ACQUITY ™ UPLC ® H-Class Bio System [Consisting of a QSM |
| | with 100 μL Mixer, TUV Detector (Flow cell: Analytical, 500 nL), |
| | FTN-SM, and CH-A heater] - Available from Waters Technologies |
| | Corporation |
| | Post-column tubing to TUV: 0.0025″ ID PEEK, 10.5″ Length |
| Data Acquisition: | MassLynx v4.1 or UNIFI v.1.8 |
| Data Analysis: | Unifi v.1.8 |
| Column: | PREMIER Peptide CSH C₁₈, 2.1 × 50 mm Column (Coated Column, |
| | coated with C2, flow path also coated with C2) |
| | ACQUITY ™ UPLC ® Peptide CSH C₁₈, 2.1 × 50 mm Column |
| | (Uncoated) - Available from Waters Technologies Corporation |
| | Both coated and uncoated columns available from Waters |
| | Technology Corporation. |
| Temperature: | 60° C., active preheater enabled |
| Seal Wash: | 10% LC-MS grade Methanol/90% 18.2 MΩ water v/v (Seal Wash |
| | interval set to 5 min) |
| Sample Manager Wash: | 10% LC-MS grade Methanol/90% 18.2 MΩ water v/v (Seal Wash |
| | interval set to 5 min) |
| Mobile Phase A: | 0.1% formic acid in 18.2 MΩ water |
| Mobile Phase B: | 0.09% formic acid in LC-MS grade acetonitrile |
| Flow Rate: | 0.2 mL/min |
| Run Time: | 30 min |
| Sample Temp.: | 8° C. |
| Sample: | 1:1:1 mixture of Angiotensin I, Enolase T37 and doubly |
| | phosphorylated insulin receptor peptide (Three Peptide Mixture) |
| | (Concentration of 20 pmol/μL) |
| Syringe Draw Rate: | 10 μL/min |
| Needle Placement: | 2.5 mm |
| Air Gaps: | Automatic |
| Data Channels: | System Pressure |
| UV Wavelength: | 214 nm |
| TUV Sampling Rate | 10 points/sec |
| Filter Time Constant | Normal |
| Data Mode | Absorbance |
| Autozero On Inject Start | Yes |
| Autozero On Wavelength | Maintain Baseline |

Experimental Settings

MS: Xevo ® G2-XS QToF 02

| | |
|---|---|
| MS Mode: | Resolution |
| MS Voltages: | Capillary: 2.5 kV; |
| | Sampling Cone: 80 V; |
| | Source Offset: 80 V |
| MS Gas Flow: | Desolvation Gas: 800 L/hr |
| MS Temperatures: | Source Temp: 120° C.; |
| | Desolvation Temp: 500° C. |
| Scan Rate: | 10 Hz |
| Acquisition Window: | 100-2000 m/z |
| Calibration: | Sodium Iodide, 100-2000 m/z |

Gradient Conditions

| | |
|---|---|
| Mobile Phase A: | 0.1% formic acid in water |
| Mobile Phase B: | 0.09% formic acid in acetonitrile |

| Time (min) | % A | % B | Curve |
|---|---|---|---|
| 0.0 | 99.5 | 0.5 | 6 |
| 12.00 | 60 | 40 | 6 |
| 14.00 | 20 | 80 | 6 |
| 14.01 | 99.5 | 0.5 | 6 |
| 17.00 | 99.5 | 0.5 | 6 |

FIG. 18 provides the Total Ion Chromatograms for the non-coated hardware (top) and the coated hardware in accordance with the present technology (bottom).

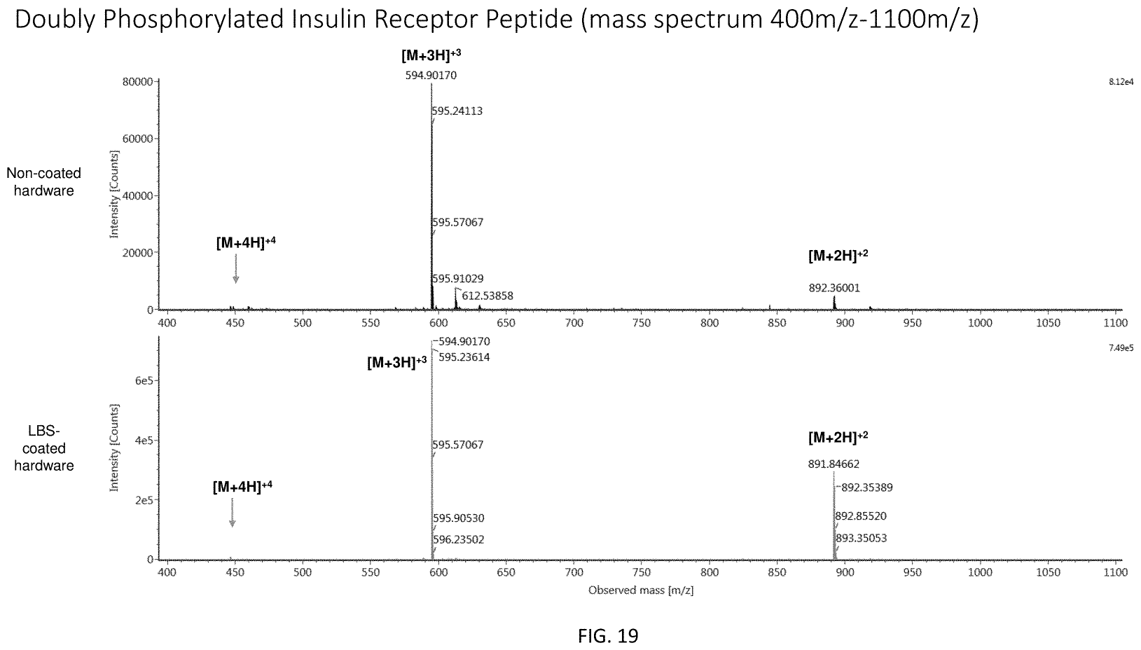

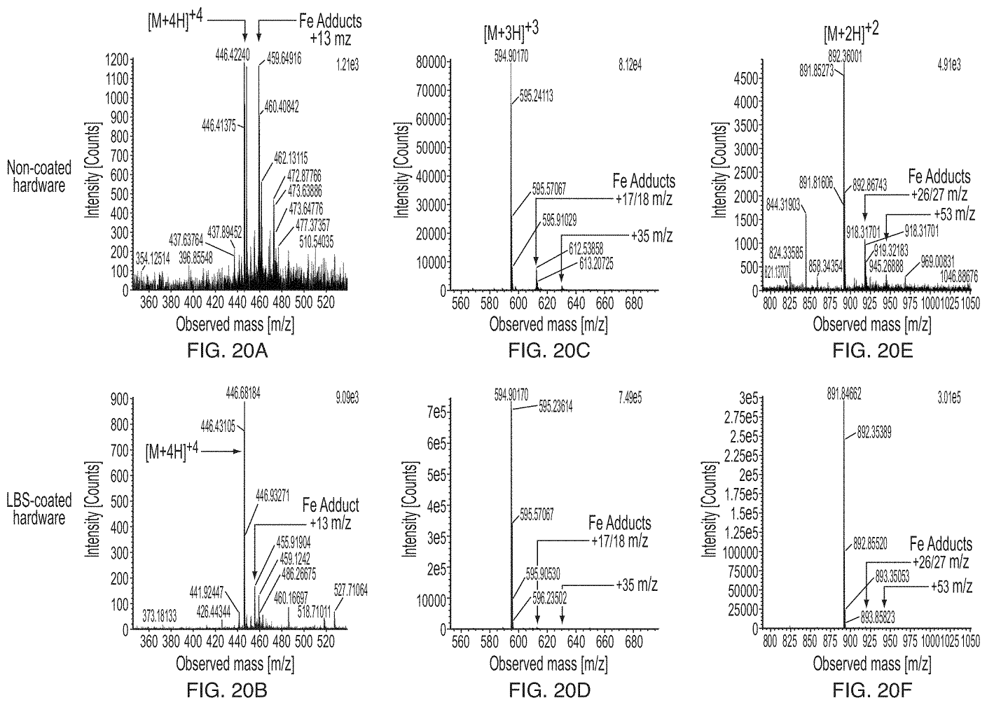

FIGS. 19 and 20 provide spectra for non-coated (top) and coated (bottom) of sample 1, doubly phosphorylated insulin receptor peptide. The results show a significant reduction in the Fe adduct.

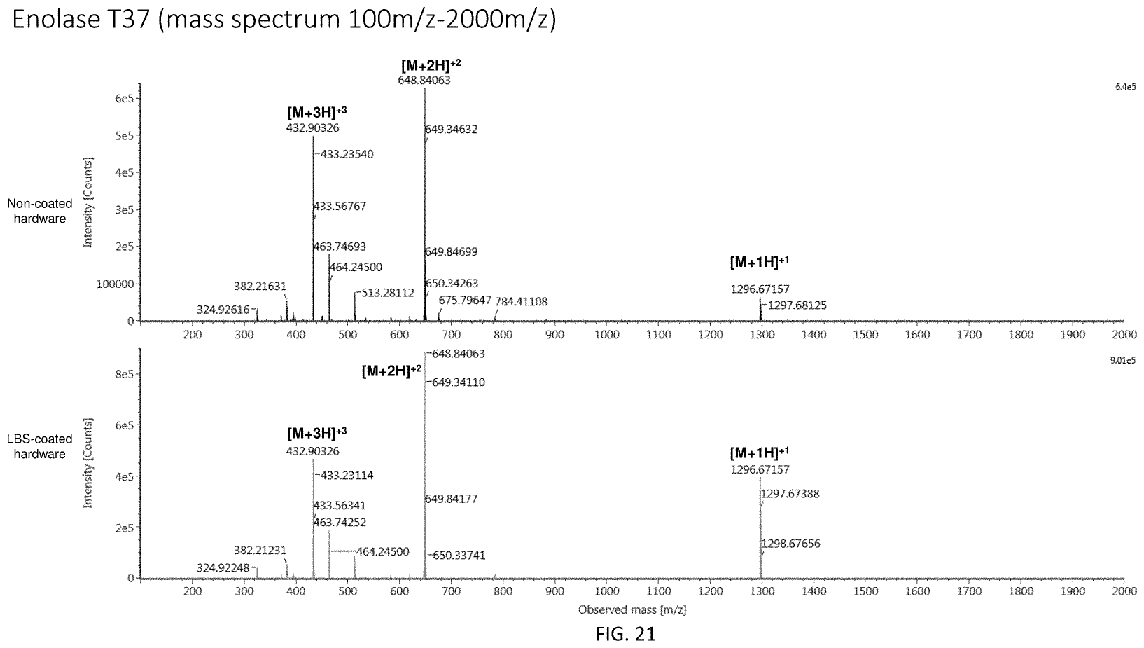

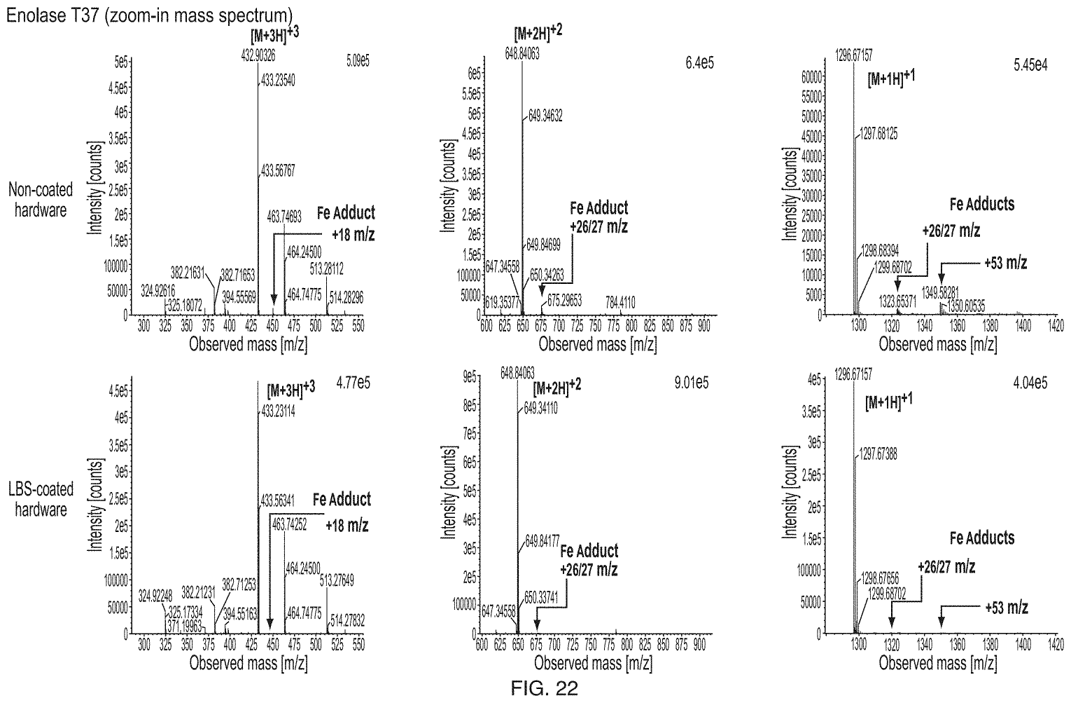

FIGS. 21-22 provide spectra for non-coated (top) and coated (bottom) of sample 2, Enolase. The results show a significant reduction in the Fe adduct.

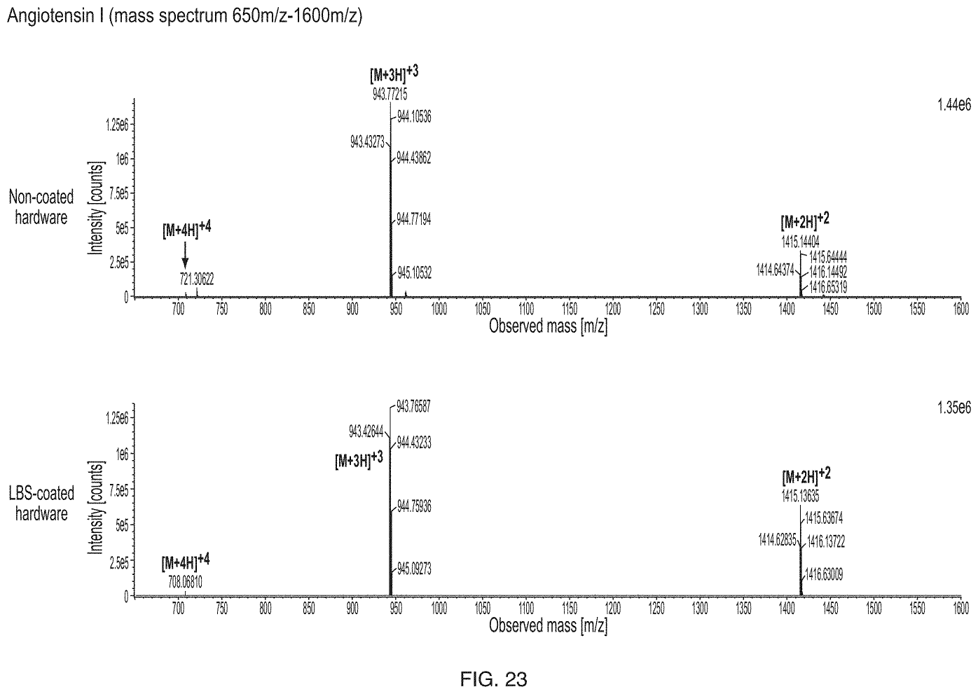

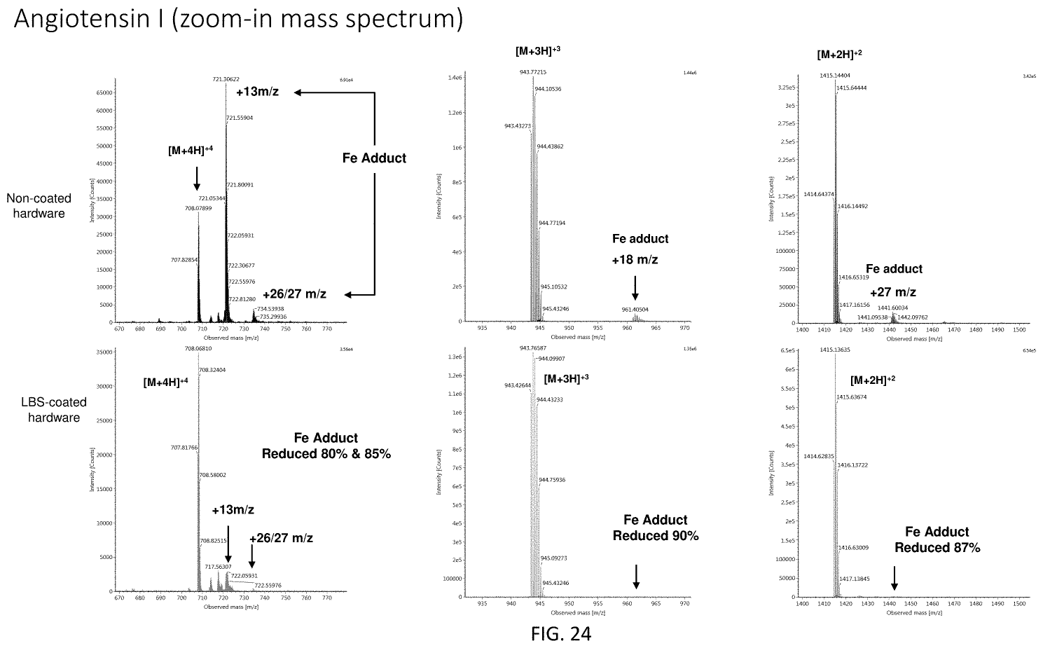

FIGS. 23-24 provide spectra for non-coated (top) and coated (bottom) of sample 3, Angiotensin I. The results show a significant reduction (i.e., 80%, 85%, 87%, and 90% reduction) in the Fe adduct.

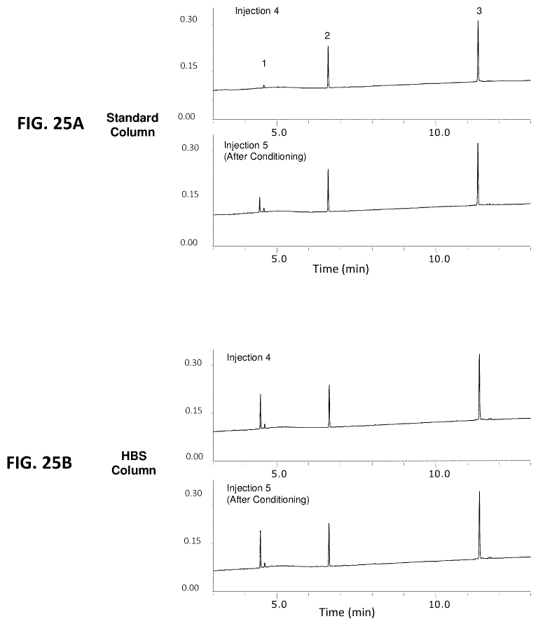

Regarding FIGS. 25A-30B, interactions of analytes with metal surfaces in High Performance Liquid Chromatography (HPLC) instruments and columns may cause a range of deleterious effects ranging from peak tailing to complete loss. These effects are due to interactions of certain analytes with the metal oxide layer on the surface of the metal components. A barrier technology has been applied to the metal surfaces in Ultra High Performance Liquid Chromatography (UHPLC) instruments and columns to mitigate these interactions. A hybrid organic/inorganic barrier based on an ethylene-bridged siloxane structure was developed for use with reversed-phase and hydrophilic interaction chromatography. The hydrolytic stability of this barrier was evaluated, and it was found to be stable from pH 1 to 12. The performance of UHPLC instruments and columns that incorporate this barrier technology have been characterized, and the results have been compared to those obtained using conventional instruments and columns. Improved performance has been demonstrated when using the barrier technology for separations of nucleotides and a phosphopeptide. The barrier technology was found to result in improved analyte recovery and peak shape, particularly when using low analyte mass loads and acidic mobile phases. Reduced abundances of iron adducts in the mass spectrum of a peptide were also observed when using UHPLC systems and columns that incorporate this barrier technology. These results suggest that this technology will be particularly impactful in UHPLC/MS investigations of metal-sensitive analytes.

For the last fifty years, stainless steel has been the most commonly used construction material for HPLC instruments and columns. The combination of high strength, compatibility with a wide range of chemicals, manufacturability and low cost make it an excellent material for many applications. However, stainless steel hardware can negatively impact the peak shape and recovery of some analytes. Analytes that show these effects typically contain functional groups such as phosphate and carboxylate groups that can form chelation complexes with iron and other transition metal ions. Stainless steel is susceptible to corrosion, particularly when exposed to acidic and/or halide-containing mobile phases, and corroded surfaces may be particularly prone to interacting with certain analytes. Alternative metals such as titanium and MP35N (a nickel-cobalt alloy) have been used for some applications because of their improved corrosion resistance, but still cause deleterious chromatographic effects for certain analytes. The engineering plastic polyether ether ketone (PEEK) has been employed to avoid these effects, but suffers from limited pressure resistance and some solvent incompatibilities. PEEK is also relatively hydrophobic and may require conditioning to avoid losses of hydrophobic analytes.

An alternative approach to mitigate interactions of analytes with metal surfaces is to add chelators such as ethylenediaminetetraacetic acid to the mobile phase or sample. Volatile chelators such as citric acid, acetylacetone and medronic acid have been used for LC/MS analyses. However, the use of chelators can negatively impact chromatographic selectivity and MS sensitivity. To address these issues, the use of a hybrid organic/inorganic barrier surface applied to the metal substrates in UHPLC instruments and columns was explored. A hybrid barrier surface based on an ethylene-bridged siloxane polymer has been found to be well-suited for reversed-phase (RP) and hydrophilic interaction chromatography (HILIC). Evaluations of the performance of UHPLC instruments and columns incorporating this hybrid barrier surface (HBS) technology relative to conventional instruments and columns were explored.

Reagents and Standards Ammonium acetate, trifluoroacetic acid (TFA), triethylamine, adenosine monophosphate (AMP) disodium salt, and adenosine triphosphate (ATP) disodium salt hydrate were obtained from Millipore-Sigma (Burlington, Mass.). Adenosine diphosphate (ADP) disodium salt hydrate and 1,1,1,3,3,3-hexafluoro-2-isopropanol (HFIP) were purchased from Acros Organics (Fair Lawn, N.J.). LC/MS grade acetonitrile was purchased from Honeywell (Muskegon, Mich.) and MS-grade formic acid (FA) was sourced from Fisher Scientific (Hampton, N.H.). Deionized water was produced using a Milli-Q system (available from Millipore-Sigma, Burlington, Mass.). Angiotensin I was acquired from Sigma Aldrich (St. Louis, Mo.) while enolase T37 and a doubly phosphorylated insulin receptor peptide with a sequence of Thr-Arg-Asp-Ile-pTyr-Glu-Thr-Asp-pTyr-Tyr-Arg-Lys (SEQ ID NO: 10) were obtained from New England Peptide, Inc. (Gardner, Mass.).

Instrumentation. A Model 190 CA Goniometer (available from ramé-hart instrument co., Succasunna, N.J.) was used to measure contact angles on silicon wafers to which the hybrid barrier surface was applied.

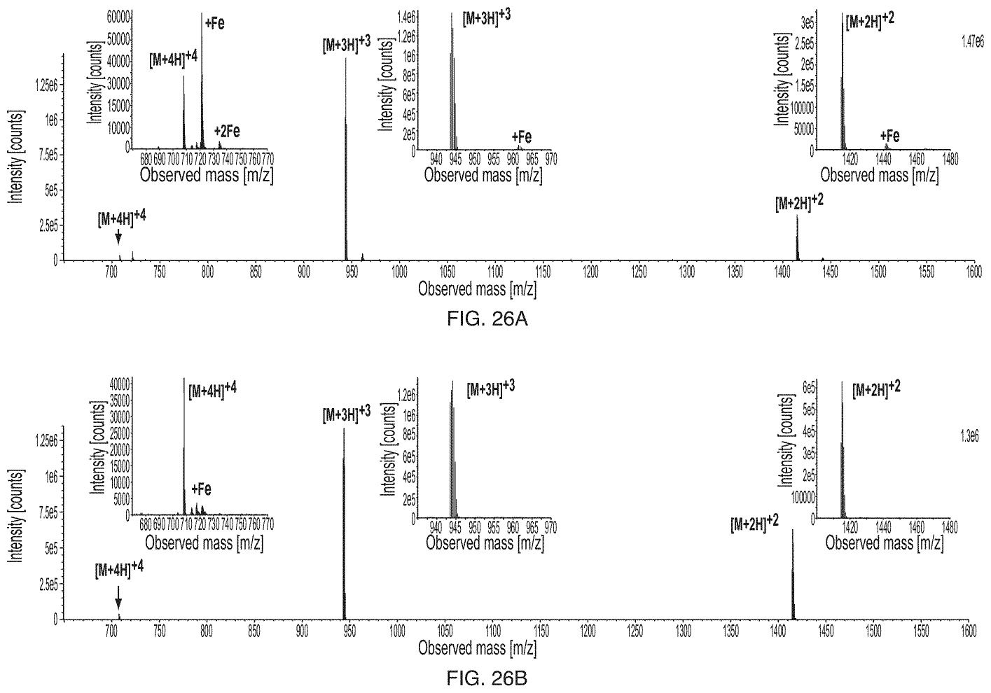

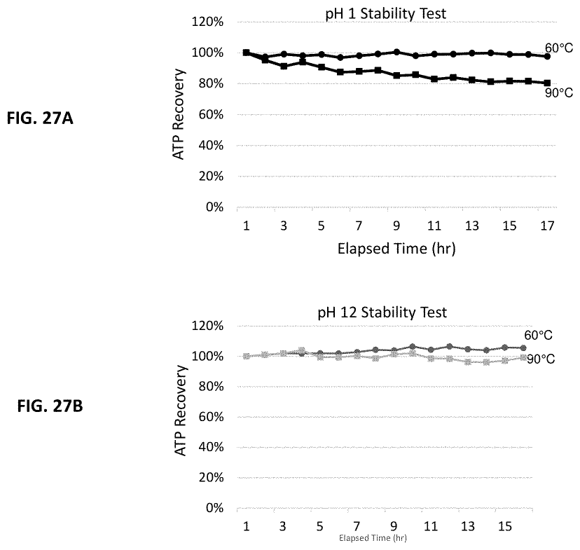

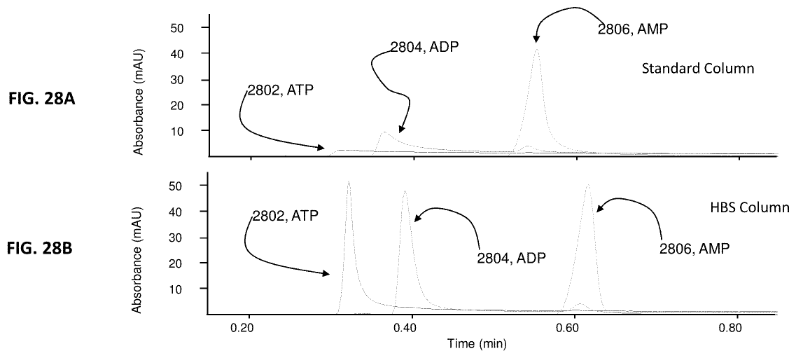

### Peptide Separations (FIGS. 25A-26B)

Chromatographic Conditions—Peptide Separations. An equimolar mixture of Angiotensin I, enolase T37, and doubly phosphorylated insulin receptor was analyzed by LC-UV using a UHPLC system such as a HBS-modified ACQUITY™ UPLC® H-Class Bio System (available from Waters Corp., Milford, Mass.) equipped with an ACQUITY™ UPLC® TUV detector (available from Waters Corp., Milford, Mass.). Separations were performed on 2.1×50 mm stainless steel columns packed with a 130 Å, 1.7 μm CSH C18 stationary phase using 0.1% formic acid (FA) in LC-MS grade water (mobile phase A) and 0.09% FA in LC-MS grade acetonitrile (mobile phase B). Separations were also performed using columns of the same dimensions constructed with hardware modified with the HBS and packed with the same batch of stationary phase as the stainless steel columns. Samples were injected at a mass load of 20 pmol and run at a temperature of 60° C., a flow rate of 0.2 mL/min, and a gradient from 0.5-40% B in 12 min, followed by 40-80% B in 2 min. Analyses were performed with UV detection at 214 nm using MassLynx™ 4.1 (available from Waters Corp., Milford, Mass.) and Empower 3 software (available from Waters Corp., Milford, Mass.) for data acquisition. UNIFI 1.8 was used for data analysis (available from Waters Corp., Milford, Mass.). A Xevo® G2-XS QTOF mass spectrometer (available from Waters Corp., Milford, Mass.) was used for MS detection, using a capillary voltage of 2.5 kV, a sampling cone and source offset of 80, a source temperature of 120° C., a desolvation temperature of 500° C., a desolvation gas flow of 800 L/h, and a collision energy of 10 eV.

To assess the initial column performance, three standard columns were compared against three columns constructed using the HBS. The same LC system modified with the HBS was used for all separations. Samples were tested before and after conditioning with 4 nmol injections of doubly phosphorylated insulin receptor peptide. Prior to conditioning, four injections of the three-peptide mixture at a mass load of 20 pmol were run, and one injection at the same mass load was analyzed after conditioning and a water blank.

Evaluation for Peptide Separations. FIG. 25A and FIG. 25B display UV chromatograms of the fourth injection (before conditioning) and fifth injection (after conditioning) of an equimolar mixture of doubly phosphorylated insulin receptor peptide (1), Angiotensin I (2), and enolase T37 (3) obtained using a standard column (FIG. 25A) or a column constructed using HBS hardware (FIG. 25B) (both 2.1×50 mm). Separations were performed with a CSH C₁₈ 130 Å, 1.7 μm, stationary phase using a flow rate of 0.2 mL/min, column temperature of 60° C., FA-modified mobile phases, and 20 pmol (25-50 ng) loads. The UHPLC system used for this experiment used parts that were treated with the HBS.

The utility of UHPLC instruments and columns constructed with HBS for the analysis of peptides was investigated. An equimolar mixture of three peptides (Angiotensin I, enolase T37, and a doubly phosphorylated insulin receptor peptide) was separated using either a standard column or a column constructed with the HBS. The columns were packed with the same batch of stationary phase and three columns of each type were tested. A UHPLC system modified with the HBS was used, with both UV and MS detection. An acetonitrile gradient was used with mobile phases containing 0.1% formic acid. As is typical for peptide separations, an elevated column temperature (60° C.) was used, along with a low flow rate (0.2 mL/min). The initial column performance was evaluated from the first four injections, using a mass load of 20 pmol (25-50 ng) of each peptide. Then a high mass load (4 nmol, 7.1 μg) of the doubly phosphorylated insulin receptor peptide was injected to condition the columns, and a fifth injection of the peptide mixture at the 20 pmol load was made to determine the impact of conditioning. Representative UV chromatograms resulting from the fourth and fifth injections are shown in FIG. 25A and FIG. 25B. While the peak areas for Angiotensin I and enolase T37 were found to be similar across the first four injections for both column types, the doubly phosphorylated insulin receptor peptide gave extremely low peak areas with the standard columns (FIG. 25A). In comparison, the HBS columns showed reproducible performance over the five injections regardless of column conditioning (FIG. 25B). The post-conditioning changes in peak area were less than 3% with the HBS columns. However, with the standard columns it was only after conditioning with a high mass load injection of peptide that the doubly phosphorylated insulin receptor peptide peak could be clearly seen. However, this peak still had only 39% of the area observed for columns constructed with the HBS, suggesting that either more conditioning is required or that full recovery cannot be achieved using these metal LC surfaces. It can be suggested that due to its two phosphate groups, the recovery of the insulin receptor peptide suffered due to a strong interaction with the positively charged oxide layer on the stainless steel surfaces.

FIG. 26A and FIG. 26B display mass spectra of Angiotensin I from a separation of an equimolar mixture of doubly phosphorylated insulin receptor peptide (1), Angiotensin I (2) and enolase T37 (3) obtained using a previously conditioned standard column (FIG. 26A) or a column constructed using the HBS (FIG. 26B) (both 2.1×50 mm). Separations were performed with a CSH C₁₈ 130 Å, 1.7 μm stationary phase using a flow rate of 0.2 mL/min, a column temperature of 60° C., FA-modified mobile phases, and 20 pmol (25-50 ng) loads. The UHPLC system used for this experiment used parts that were treated with the HBS.

Mass spectrometric (MS) data was also obtained for the three-peptide mixture as separated using the conditioned columns and performed with electrospray ionization and high sensitivity quadrupole time-of-flight instrumentation. At first glance, the results from the total ion current chromatograms appeared to support the data acquired using UV detection, where there is little difference between the columns with respect to Angiotensin I and enolase T37. However, upon further investigation, there is contrast in the mass spectra, where higher quality MS data was obtained using the HBS columns. Separations using standard columns yielded a relatively high ion signal for iron adducts. As exemplified in the mass spectra of Angiotensin I (FIG. 26A and FIG. 26B), these separations can be populated with iron ions that are leached from the stainless steel surface such that an iron adducted peak can become an abundant feature (FIG. 26A). The level of iron adducts in the 3+ charge state of Angiotensin I was 5.9%. For the 2+ charge state, the level of adduction was 9.5%. For the 4+ charge state, the abundance of the iron adducted peak was greater than that of the primary peak. In comparison, separations performed using columns constructed with the HBS showed 80-90% reduced abundances of iron adducts (FIG. 26B). Interestingly, the charge state distribution for columns with the HBS gave higher relative abundances for the lower charge states, suggesting that iron adduction affects ionization and forces analytes to occupy higher charge states. For example, the relative abundance of the 2+ charge state of angiotension I as given by standard columns was 19.5% versus 29% for the columns with the HBS. The presence of iron adducts makes the mass spectra more difficult to interpret due to distortions in the relative abundances of protonated species and increased spectral crowding.

### Hydrolytic Stability Studies and Nucleotide Separations (FIGS. 27A-30A)

Hydrolytic Stability Studies. The stability studies were performed using a Waters (Milford, Mass.) ACQUITY™ I-Class system consisting of a binary solvent manager, fixed loop sample manager, CH-A column heater, and a TUV detector. To eliminate the system as a variable in the testing, a PEEK needle, PEEK sample loop, and an active preheater with the hybrid barrier surface (HBS) was used. A custom fixture was designed to allow testing of individual frits without a column. Tests were performed on 0.2 μm porosity grade titanium frits with a diameter of 4.6 mm and a thickness of 1.5 mm, to which the HBS was applied. A flow rate of 0.2 mL/min and a temperature of either 60 or 90° C. was used. ATP was monitored using absorbance at a wavelength of 260 nm. For the acid stress testing the following test sequence was used; flow 1% TFA (pH 1) for 1 hour, flow 50/50 (v/v) methanol/water for 10 minutes to remove adsorbed TFA from the system, flow aqueous 10 mM ammonium acetate pH 6.8 for 10 minutes to raise the pH to be suitable for testing with ATP, then inject a water blank followed by 0.2 μL of 50 μg/mL ATP (prepared in aqueous 10 mM ammonium acetate pH 6.8). This sequence was repeated for 16 hrs. For the accelerated base stress testing the following test sequence was followed; flow aqueous 10 mM sodium hydroxide (pH 12) for 1 hour, flow aqueous 10 mM ammonium acetate pH 6.8 for 10 minutes to raise the pH to be suitable for testing with ATP, then inject a water blank followed by 0.2 μL of 50 μg/mL ATP (prepared in aqueous 10 mM ammonium acetate pH 6.8). This sequence was repeated for 16 hrs.

Chromatographic Conditions—Nucleotide Separations. Instruments used include a UHPLC system such as a ACQUITY™ UPLC® H-Class instruments (available from Waters Corp., Milford, Mass.) equipped with a Quaternary Solvent Manager (QSM), a Flow-Through Needle Sample Manager (SM-FTN), a CH-A and ACQUITY™ UV detectors, either a photodiode array (PDA) detector or a tunable UV (TUV) detector. Both standard instruments and a modified version using components treated with the HBS were used. Isocratic separations of ATP, adenosine diphosphate (ADP), and adenosine monophosphate (AMP) were achieved using aqueous 10 mM ammonium acetate mobile phases, at a flow rate of 0.5 mL/min. Unless noted otherwise, the pH of the mobile phase was 6.8. The samples, freshly prepared daily in 100% water, were injected onto a UHPLC such as an ACQUITY™ UPLC® BEH C18 130 Å, 1.7 μm, 2.1×50 mm columns at 30° C. (available from Waters Corp., Milford, Mass.). Separations were also performed using columns of the same dimensions constructed with hardware modified with the HBS and packed with the same batch of stationary phase. The injected masses ranged from 20-100 ng of each nucleotide. Columns were equilibrated with the isocratic condition before the injections. All tests were carried out using new columns. The UV response at 260 nm was recorded using an Empower 3 or a MassLynx™ 4.2 Chromatography Data System (available from Waters Corp., Milford, Mass.).

Characterization of the Hybrid Barrier Surface. The barrier is an ethylene-bridged siloxane polymer ((O_{1.5}SiCH₂CH₂SiO_{1.5})ₙ) that is formed on metal substrates using a vapor deposition process. The chemical composition of this barrier is related to that of ethylene-bridged hybrid (BEH) chromatographic particles. This layer has a static water contact angle of approximately 30°, significantly lower than the 70-90° reported for PEEK. This indicates that the hybrid barrier surface (HBS) is significantly more hydrophilic than PEEK, making it less prone to hydrophobic adsorption. The vapor deposition technique is able to provide an effective barrier even on high aspect ratio substrates, such as tubing with an internal diameter of 100 μm and a length of 368 mm. This makes it possible to implement the technology across diverse types of LC hardware and column components.

To characterize the effectiveness of this barrier in mitigating interactions of metal-sensitive analytes with metal substrates, the recovery was measured of a low mass load of adenosine triphosphate (ATP), which is known to show severe losses when chromatographed using metal surfaces. Column frits with and without the HBS were tested using a UHPLC system in which PEEK tubing was used in place of stainless steel tubing. The mobile phase for these experiments was aqueous 10 mM ammonium acetate (pH 6.8). A UV detector was used to quantify the ATP. The area of the peak was compared to that obtained without the frit, allowing the recovery to be calculated. Titanium frits with a diameter of 4.6 mm, a thickness of 1.5 mm and a porosity grade of 0.2 μm showed an ATP recovery of less than 5% for a 10 ng injection. After the HBS was applied, the ATP recovery average increased to 99.7% with a standard deviation of 1.6% for 32 frits, each prepared with an independent application of the HBS. This demonstrates the effectiveness and reproducibility of the HBS.

FIG. 27A and FIG. 27B display the accelerated stability test results for a 4.6 mm diameter 0.2 μm titanium frit with the HBS. FIG. 27A displays the pH 1 tests that used 1% TFA (aq), and FIG. 27B displays the pH 12 tests that used 10 mM NaOH (aq). ATP recoveries were determined using UV detection and an aqueous 10 mM ammonium acetate (pH 6.8) mobile phase.

A similar test was also used to characterize the hydrolytic stability of the HBS, using accelerated conditions. Aqueous solutions containing 1% trifluoroacetic acid (TFA) (pH 1) or 10 mM NaOH (pH 12) were flowed through titanium frits with the HBS at 60 and 90° C. After one hour, the mobile phase was changed to 10 mM ammonium acetate (pH 6.8) and one 10 ng ATP injection was made. After the ATP injection, the mobile phase was changed back to 1% TFA or 10 mM NaOH and the sequence was repeated. The results of these tests are shown in FIG. 27A and FIG. 27B. In the pH 12 tests, no significant change in ATP recovery was observed after 16 hours at both 60 and 90° C. The pH 1 test at 60° C. also showed no significant change in recovery, while the 90° C. test resulted in a 20% decrease. These results indicate that the HBS has stability that is similar to that of C18-bonded BEH particles, which are recommended for use over the pH range of 1 to 12.

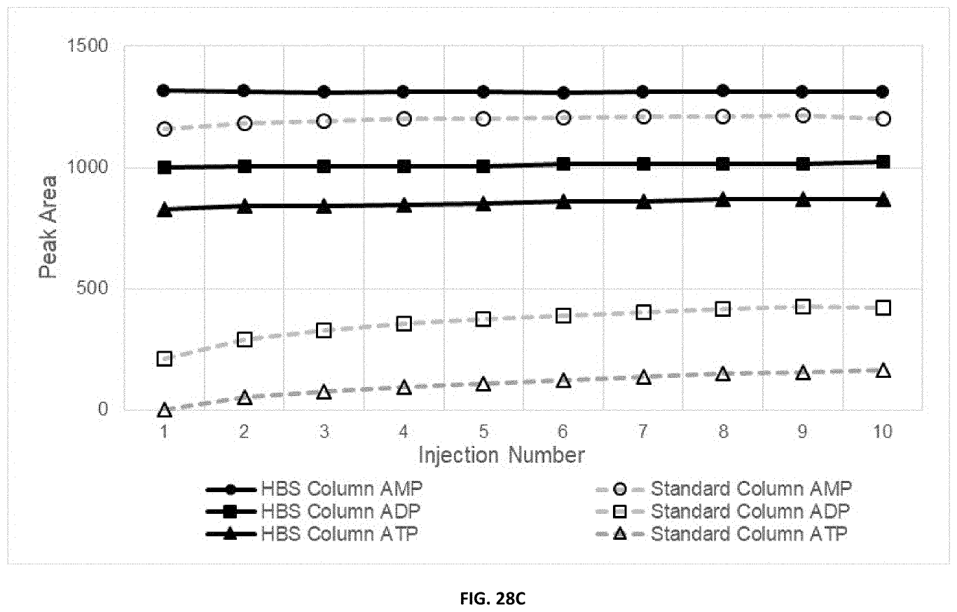

Characterization of Columns with the HBS. FIG. 28A and FIG. 28B display a comparison of the separation of AMP (2806), ADP (2804) and ATP (2802) using a standard BEH C₁₈ column and a BEH C₁₈ column constructed with hardware treated with the HBS. FIG. 28C displays ten sequential injections of the mixture (100 ng of each analyte) that were made. FIG. 28A displays a chromatogram from the fifth injection on the standard column. FIG. 28B displays a chromatogram from the fifth injection on the HBS column. FIG. 28C displays a plot of recovery of each analyte vs. injection number. The UHPLC system used for this experiment used parts that were treated with the HBS. The mobile phase was aqueous 10 mM ammonium acetate pH 6.8, and detection was by absorbance at 260 nm.

The separations of ATP, ADP and AMP achieved using a standard column versus a column constructed using hardware treated with the HBS were compared. The UHPLC system used for this experiment was equipped with components that were treated with the HBS. The mobile phase was aqueous 10 mM ammonium acetate (pH 6.8). A series of ten injections was made of a solution containing 20 ng of each nucleotide. A tunable UV detector (λ=260 nm) was used for these experiments. FIG. 28A and FIG. 28B are chromatograms for the fifth injections obtained using a standard 1.7 μm BEH C₁₈ 2.1×50 mm column (FIG. 28A) and a column containing the same packing material but using column hardware treated with the HBS (FIG. 28B). FIG. 28C shows the peak areas determined for these analytes over the ten injections. The results show that the standard column exhibited low peak areas and severe tailing for ADP and ATP. The peak areas increased over the series of injections, but after ten injections failed to reach the areas obtained using the HBS column. In contrast, the column constructed using hardware with the HBS gave consistent peak areas across the ten injections for all three analytes.

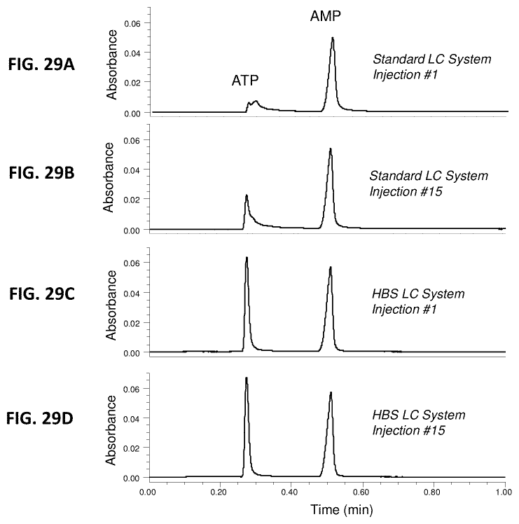

Characterization of a UHPLC Instrument with the HBS. FIG. 29A, FIG. 29B, FIG. 29C, and FIG. 29D show a comparison of the separation of AMP and ATP using a standard UHPLC system (FIG. 29A and FIG. 29B) and a UHPLC system constructed using parts treated with the HBS (FIG. 29C and FIG. 29D). A 1.7 μm BEH C₁₈ 2.1×50 mm column constructed with hardware treated with the HBS was used. The mobile phase was aqueous 10 mM ammonium acetate pH 6.8, and detection was by absorbance at 260 nm. Fifteen sequential injections of the mixture (20 ng each analyte) were made. Chromatograms are shown for injection 1 (FIG. 29A and FIG. 29C) and injection 15 (FIG. 29B and FIG. 29D) for both UHPLC systems.

To evaluate the performance of a UHPLC system in which the parts were treated with the HBS, the separation of a mixture of AMP and ATP was used. The results were compared to those obtained using a standard metal surface UHPLC system. A 1.7 μm BEH C₁₈ 2.1×50 mm column constructed using hardware treated with the HBS was used for this experiment. Fifteen sequential injections of a mixture containing 20 ng of each analyte were made. The results for the first and last injections are shown in FIGS. 29A-29D. The standard UHPLC system initially gave a severely tailing peak for ATP, with the peak area gradually increasing with the injection number. The AMP peak showed a more consistent peak area and shape throughout the fifteen injections. In contrast, the UHPLC system treated with the HBS showed consistent peak area and shape for both ATP and AMP throughout the fifteen injections.

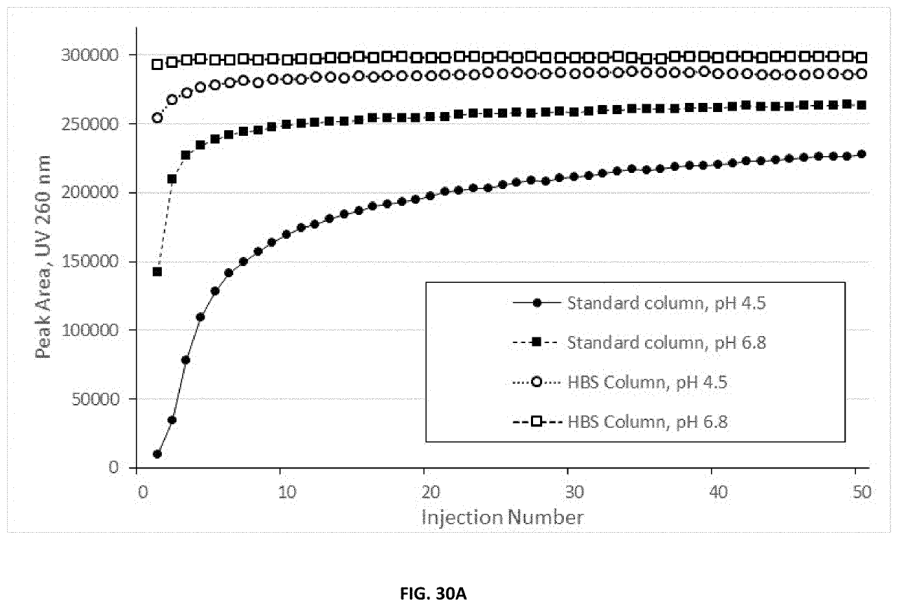

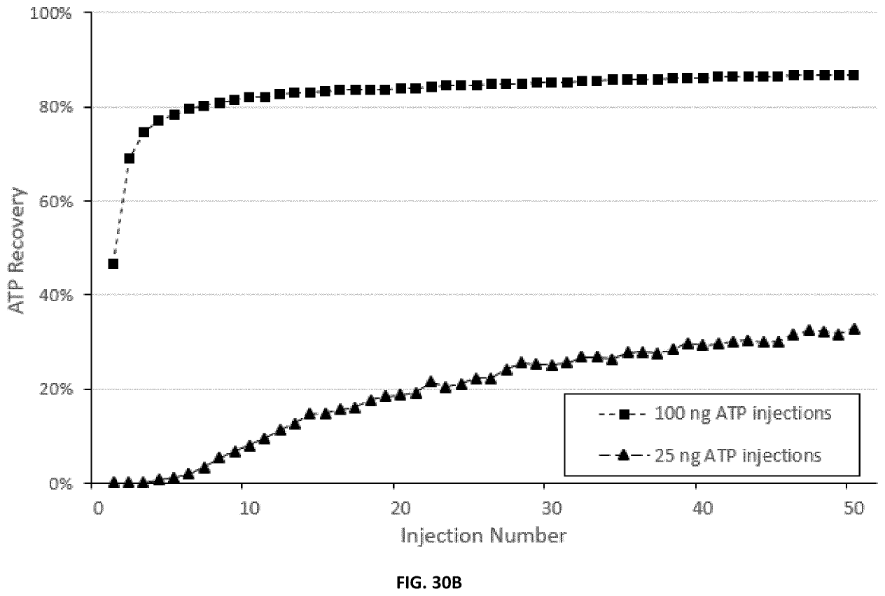

pH Dependence of ATP Losses. FIG. 30A displays a comparison of the peak area of ATP vs. injection number using different mobile phase pH values for a standard ACQUITY™ BEH C₁₈ column and a BEH C₁₈ column constructed with hardware treated with the HBS. The mobile phases contained 10 mM ammonium acetate, with the pH adjusted to either 4.5 or 6.8. Detection was by absorbance at 260 nm. Fifty sequential injections of 100 ng of ATP were made. The UHPLC system used for this experiment used a flow path treated with the HBS. FIG. 30B displays ATP recovery vs. injection number using different injection loads for a standard ACQUITY™ BEH C₁₈ column (available from Waters Corp., Milford, Mass.). The mobile phase was aqueous 10 mM ammonium acetate pH 6.8, and detection was by absorbance at 260 nm. Fifty sequential injections of 100 or 25 ng of ATP were made. Recoveries were calculated as the ratio of the peak area observed with the column vs. that obtained without it. The UHPLC system used for this experiment used parts that were treated with the HBS.

The dependence of the peak area for ATP on mobile phase pH, using both a standard column and a column constructed using hardware treated with the HBS was investigated. The UHPLC system used for this experiment had components that were treated with the HBS. The mobile phases all contained aqueous 10 mM ammonium acetate, with the pH adjusted by adding acetic acid or ammonium hydroxide. A series of 50 sequential injections of 100 ng of ATP were made on a previously unused standard 1.7 μm BEH C₁₈ 2.1×50 mm column, and on a column containing the same packing material but using column hardware treated with the HBS. The results are shown in FIG. 30A. Using a pH 4.5 mobile phase, the standard column caused almost complete loss of ATP in the initial injections. As more injections were made, the peak area gradually increased, but never reached the expected area, even after 50 injections. Using a pH 6.8 mobile phase, the standard column showed a ca 50% ATP loss in the first injection, with a gradual increase in peak area with injection number. When a pH 8.5 mobile phase was used with a standard column, the ATP peak area was slightly low (ca 5% loss) in the first injection, but quickly reached the expected area. In contrast, columns using hardware treated with the HBS showed much more consistent ATP peak areas, regardless of mobile phase pH.

Mass Load Dependence of ATP Recovery. The dependence of the recovery of ATP on the mass injected for a standard column was investigated. The UHPLC system used for this experiment was equipped with components that were treated with the HBS. For this experiment an aqueous 10 mM ammonium acetate (pH 6.8) mobile phase was used. Fifty sequential injections of ATP were made on a previously unused standard 1.7 μm BEH Cis 2.1×50 mm column. Two ATP injection masses were compared, 25 and 100 ng. The recoveries were calculated as the ratio of the peak areas observed with the column to the areas without the column. The results are shown in FIG. 30B. At the lower loading, ATP was not detected in the first three injections, and the recovery increased very gradually, only reaching 35% after 50 injections. At the higher loading, the recovery for the first injection was 45%, increased to 80% by the seventh injection, then slowly continued to increase, reaching 85% after 50 injections. These results shows that analyte losses are most severe for low on-column mass loadings. These results also suggest that the interaction of ATP with the metal surfaces is partially reversible, since the recovery never reaches 100%, even after 50 injections at the 100 ng loading. It appears that some of the adsorbed analyte is released as the mobile phase continually flows through the column. This result indicates that attempting to condition an HPLC system and column by making injections of the analyte before the sample to be analyzed may fail because the analyte adsorbed in the conditioning injections may be partially eluted before the sample is injected.

The HBS technology described here provides a means to improve UHPLC analyses of analytes that interact with metal surfaces. High recoveries and more symmetric peaks demonstrated to be obtainable using UHPLC systems and columns that incorporate this technology, even for challenging analytes such as ADP, ATP, and a doubly phosphorylated peptide. Other phosphorylated analytes that benefit from this technology include phosphoglycans and sugar phosphates. In addition, significant benefits have been observed for analytes containing multiple carboxylate groups, such as citric acid and acidic peptides.

HBS UHPLC systems and columns have been shown to give the biggest improvement over their standard counterparts at low mass loads. This suggests that methods employing UHPLC/MS will benefit greatly from this technology, particularly when trace level quantitative measurements are needed. The reduction in the occurrence of iron adducts noted in the mass spectra of the peptides is particularly important in studies using library matching. Work is in progress to further demonstrate the range of applications that benefit from this technology.

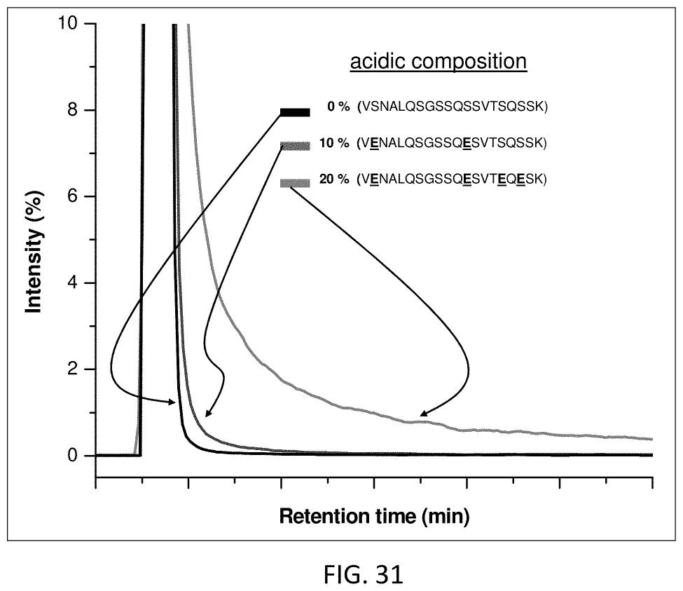

FIG. 31 displays a synthetic acidic peptide ladder used to evaluate tailing for 3 synthetic peptides manufactured with 0, 2, and 4 glutamic acid (E) residues representing 0%, 10%, and 20% acidic content by composition. Under isocratic conditions (mobile phase A: 89%; and mobile phase B: 11%), tailing was observed to increase significantly for peptides containing multiple acidic residues.

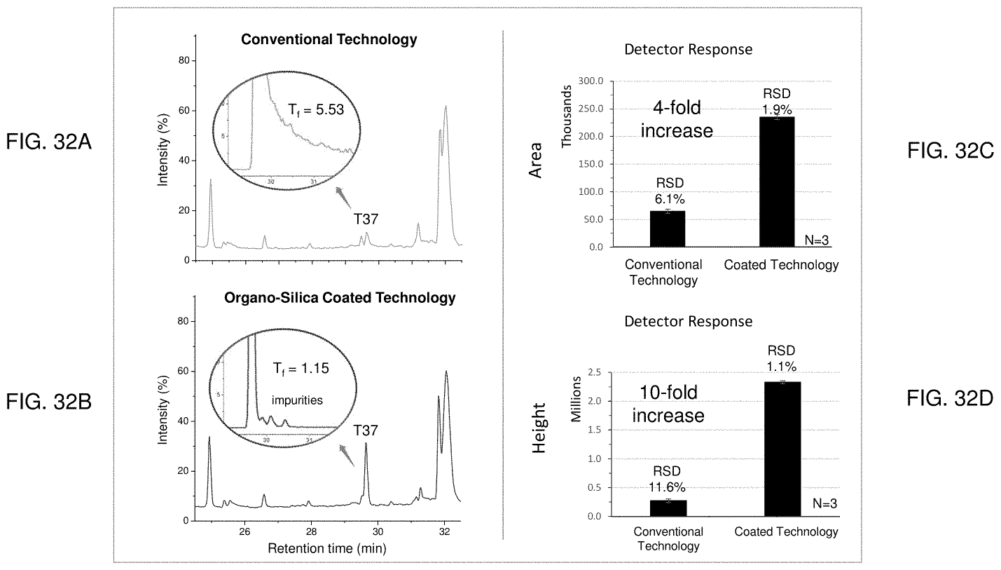

FIG. 32A and FIG. 32B display recovery of a T37 peptide fragment from a tryptic digest of the NIST reference mAb standard that was evaluated for a peptide map performed on a conventional column (stainless-steel; FIG. 32A) as well as an alkylsilyl coated column (ACQUITY™ PREMIER column available from Waters Technologies Corp. Milford, Mass.) (FIG. 32B).

FIGS. 31 and 32A-32D display increasing recovery and reproducibility of acidic peptides in RPLC-based assays by reducing analyte/surface interactions. Metal-ion mediated adsorption of sensitive analytes in LC-based assays can negatively impact data quality and assay robustness. Peptide columns including a coating in accordance with the present technology can minimize analyte/surface interactions while increasing reproducibility, enhancing peak shape (e.g., decreasing peak width and/or peak tailing), and increasing recovery of sensitive analytes.

Analyte/surface adsorption in liquid chromatography can be a contributing factor in poor peak shape, tailing, and diminished recovery of compounds in LC-based techniques. Metal-ion mediated adsorption has been identified as an adsorption mechanism for analytes that exhibit Lewis acid/base characteristics. Without wishing to be bound by theory, the analytes bearing electron rich moieties (such as phosphate groups, uncharged amines, and deprotonated carboxylic acids) act as Lewis Bases, which can adsorb in a non-covalent manner to electron deficient sites on the metal surface which act as a Lewis Acid. This reaction is evident in peptide analyses. For example, peptide fragments containing aspartic acid (D) or glutamic acid (E) residues can interact with metal surfaces, which can exacerbate adsorption characteristics resulting in increased tailing and reduced sensitivity of analytes prone to metal-ion mediated adsorption as shown in FIG. 31.

The coated column technology of the present disclosure offers a solution to mitigate metal-ion mediated adsorption without the need to alter sample matrix, mobile phase composition, or incorporate passivation protocols. This is accomplished through the application of practices and knowledge of organosilica chemistry to introduce a column that sets a precedence for inert characteristics toward metal sensitive analytes. In some embodiments, the alkylsilyl coatings applied to the columns allows for improved analysis of metal sensitive analytes. The columns including a coating in accordance to the present technology (e.g., columns including an organosilica coating) can increase productivity in the lab and mitigate risk during the development and manufacturing of pharmaceutical drug products.

Table 5 displays the following experimental parameters for FIGS. 31 and 32A-32D.

TABLE 5
Experimental Parameters

| | |
|---|---|
| LC System: | ACQUITY ™ H-Class Binary Bio PLUS |
| Sample: | Waters mAb Tryptic Digestion Standard, 0.2 mg/mL |
| Detection: | TUV, 10 mm Analytical FC, λ = 214 nm |
| Column(s): | Uncoated Column: CSH 130 Å C18 column (2.1 × |
| | 100 mm, 1.7 μm p/n 186005297) |
| | Organosilica Coated Column: ACQUITY ™ |
| | PREMIER Peptide CSH 130 Å C18 column |
| | (2.1 × 100 mm, 1.7 μm p/n 186009461) |
| Column Temp.: | 60° C. |
| Mobile Phase A: | H₂O, 0.1% Formic acid |
| Mobile Phase B: | Acetonitrile, 0.1% Formic acid |
| Gradient: | 0.68 % B/min, 0.200 mL/min |

Deamidation of asparagine to aspartic acid and iso-aspartic acid is a post-translational modification of monoclonal antibodies that can be been correlated to drug efficacy. The “PENNYK” T:37 peptide (SEQ ID NO: 2) (sequence: GFYPSDIAVEWESNGQPENNYK (SEQ ID NO: 1), underlined letters=acidic) is of notable interest in that it is a monitored Fc domain peptide, which is known to be susceptible to post translation modifications such as deamidation and contains 4 “acidic” residues. As shown in FIG. 32A, a tailing factor (T_{f}) of 5.53 was observed for the native PENNYK peptide (SEQ ID NO: 2) using a conventional column (i.e., a column with no coating) which prevented detection of the closely eluting associated impurities. In contrast, when the same separation was performed using the organosilica coated column (FIG. 32B), tailing was reduced by 79% (T_{f}=1.15) allowing for the closely eluting impurities to be resolved from the native peak.

The observed performance gain afforded by the coated column in accordance with the present technology due to the reduced tailing resulted in a 4-fold increase peak area (FIG. 32C) and a 10-fold increase in detector response (FIG. 32D). In addition to providing more information in the interpretation of data through increased recovery and peak shape, the organosilica coated column was observed to reduce assay variability. As shown in FIG. 32D, the observed increase in recovery resulted in an approximately 90% reduction in response variability (height), which was observed with calculated R.S.D. being reduced from 11.6% to 1.1% for 3 replicate injections when using the organosilica coated column (commercially available from Waters Technologies Corp.).

Analyte/surface adsorption in liquid chromatography can lead to increased assay variability, reduced assay sensitivity, and miss-interpretation of sensitive analytes. Organosilica coated columns in accordance with the present technology can minimize analyte/surface interactions while increasing reproducibility, peak shape, and recovery of sensitive analytes during the development and manufacturing of pharmaceutical drug products. In some examples, another peptide can be used such as peptide T43p, (sequence: VNQIGpTLSESIK (SEQ ID NO: 15)), monoisotopic mass 1368.6776 Da.

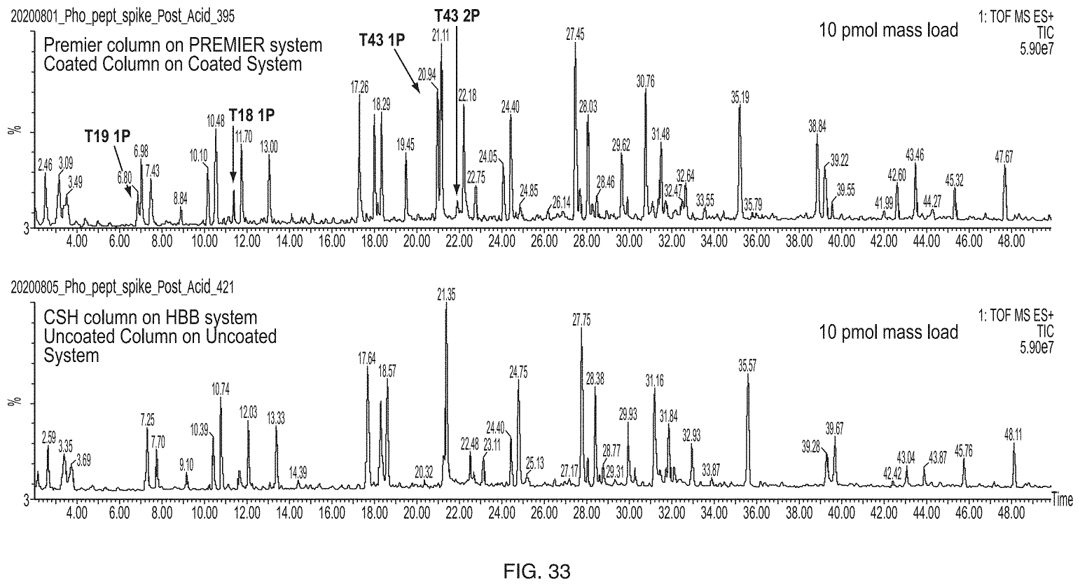

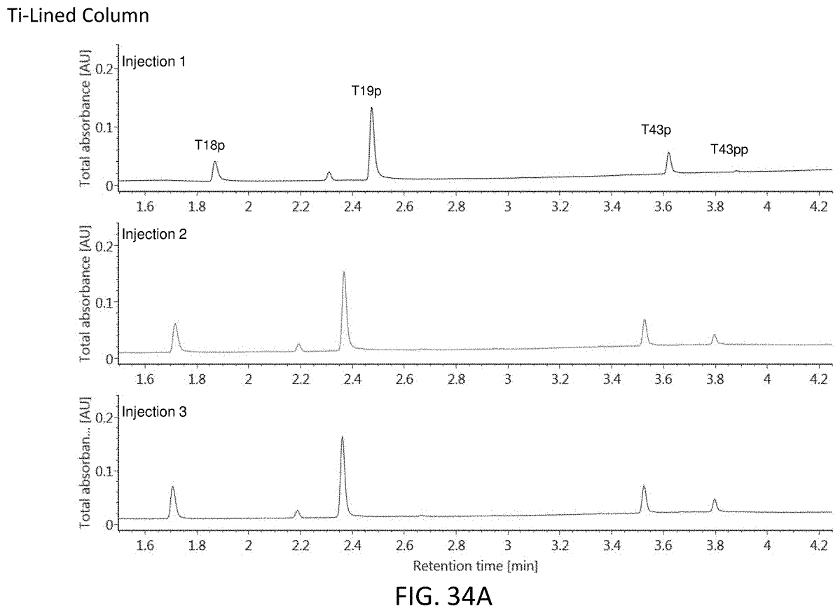

FIG. 33 displays a phosphopeptide application to demonstrate the performance differences between an organosilica coated peptide and a commercially available column (uncoated column). Phosphopeptides contain anionic phosphate groups that can adsorb to the electron-deficient surfaces of metals. A mixture of phosphopeptides was used that contains four synthetic enolase phosphopeptides: three phosphopeptides that are singly phosphorylated (T19 1P, T18 1P, and T43 1P) and one phosphopeptide that is doubly phosphorylated (i.e., two phosphate moieties) (T43 2P (also known as T43 PP)).

For the singly phosphorylated phosphopeptides, the system and column adsorb approximately 13% of peptides. For the doubly phosphorylated phosphopeptide (T43 2P), the system and column adsorbs almost all of peptides at the 10 pmol mass load. Table 6 compares the results for 10 pmol mass load for the organosilica coated peptide column with and a commercially available uncoated column (e.g., conventional column without organosilica coating). To obtain the area ratio and the height ratio, the result from the organosilica coated column is divided by the result from the commercially available column.

TABLE 6
10 pmol mass load results

| Peptide | Area Ratio | Height Ratio |
|---|---|---|
| T19 1P | 0.84 | 0.24 |
| T18 1P | 0.95 | 0.62 |
| T43 1P | 0.83 | 0.12 |
| T43 2P | 0.00 | 0.00 |

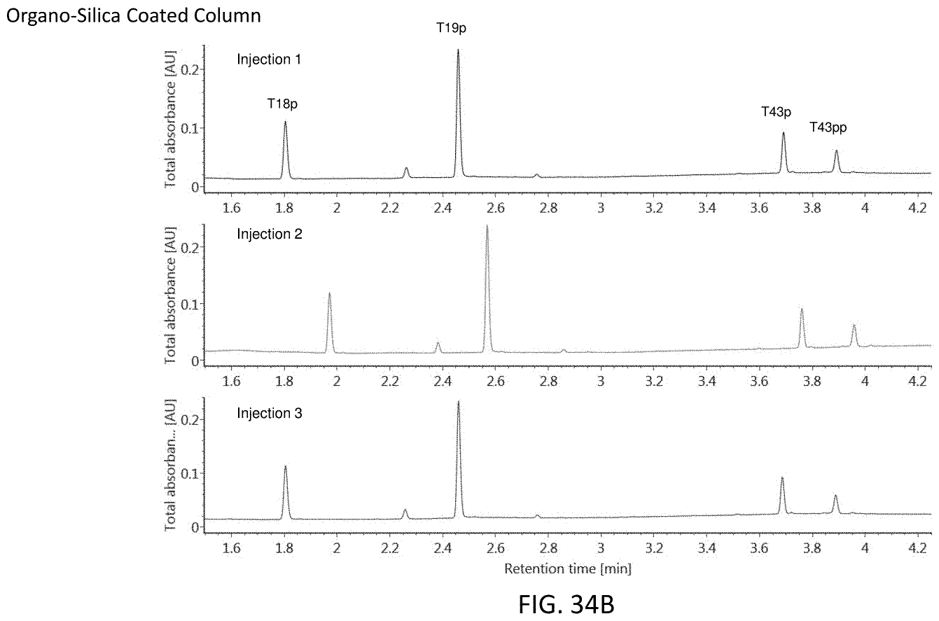

FIGS. 34A and 34B compare the chromatographic performance of a organosilica coated peptide C18 column versus a titanium-lined C18 column technology. Specifically, FIGS. 34A and 34B compare the chromatographic performance of the organosilica coated peptide CSH column (commercially available from Waters Technologies Corp.) to the Phenomenex bioZen™ Peptide PS-C18 column (available from Phenomenex Co., Torrance, Calif.). The Phenomenex column is a titanium-lined C18 column. FIG. 34A is a TIC chromatogram for the first three injections using the bioZen™ column. FIG. 34B is a TIC chromatogram for the first three injections using the organosilica coated peptide column.

For FIGS. 34A and 34B, there is little to no difference in the abundance of charge states or adduct formation between the bioZen™ and organosilica coated columns when looking at the peptide MS spectra.

The bioZen™ column is a positively charged stationary phase intended to be a bioinert column for peptide separations. The bioZen™ column is a titanium-lined column with titanium frits.

Regarding the packed bed stability of both columns, the bioZen™ and PREMIER columns (e.g., organosilica coated columns) are both rated to a maximum operating pressure of 15,000 psi.

The experimental conditions for FIGS. 34A and 34B include:

### Columns

Ti-Lined Column: 2.1×50 mm Phenomenex bioZen™ 1.4μ Peptide PS-C18 (H18-167549)
Organosilica Coated Column: 2.1×50 mm PREMIER Peptide CSH C18 1.7 μm (01622933750K02, batch 0162)
   H-Class Bio 03 modified with a hybrid organic/inorganic flow path and coupled to a Xevo® G2-XS QToF
   Mobile Phase A: 0.1% Formic Acid, milli-Q water

### Mobile Phase B: 0.075% Formic Acid, Acetonitrile (Optima Grade)

Flow Rate: 0.60 ml/min.

### Gradient: 0.7% to 25% Acetonitrile

Gradient Time: 5 minutes with a 3 min equilibration at the end

### Column Temp.: 60° C.

### TUV Detector: 220 nm

### Sample: Waters MassPREP™ Phosphopeptide Standard Enolase, p/n 186003285, Lot W19031802

1 nmol each of T19P, T18P, T43P, T43PP
   Reconstitution: 50 μl of 0.1% F.A. (formic acid) in water per vial, combine 3 vials into Q-Sert vial (Waters P/N 186001126C) per lot

### Injection Volume: 10 μL

For the four phosphopeptide standard (T19P, T18P, T43P, and T43PP), the UV peak area remained relatively linear across the first three injections for the organosilica coated column at 400,000. In contrast, the titanium-lined column increased for each successive injection for the first three injections (1^{st} injection: ˜260,000; 2^{nd} injection: ˜340,000; and 3^{rd} injection: ˜360,000). For the third injection of each column, the titanium-lined column has 9.7% lower summed 4-peptide peak area than the organosilica coated column.

For the UV Peak Capacity of the four phosphopeptide standard (T19P, T18P, T43P, and T43PP), the organosilica coated column and the titanium-lined column remained relatively constant across the first injections; the organosilica coated column had a UV peak capacity of ˜350 and the titanium-lined column had a UV peak capacity of ˜280. For the third injection of each column, the titanium-lined column had a 18.7% lower peak capacity than the organosilica coated column.

To summarize the results, the titanium-lined (bioZen™ from Phenomenex) column showed minimal recovery of the T43PP upon first injection. Injections 1-3 showed large improvements in recovery, suggesting that further conditioning may be required for the titanium-lined column. There was variable peak recovery when using the titanium-lined column across 10 injections.

The organosilica coated column performance showed increased peak capacity and decreased tailing versus the titanium-lined column upon the initial three injections.

Compared to the titanium-lined column, the organosilica coated column had peak capacities that were around 20% higher and lower abundant species that were better resolved. In addition, the organosilica coated column was more mechanically stable than the titanium-lined column.

The above aspects and features of the present technology provide numerous advantages over the prior art. In some embodiments, there are numerous benefits incorporating the coating through the column (and in some embodiments through the entire fluidic pathway from sample reservoir to the detector) to define a LBS (e.g., an organosilica coated surface). For example, the present disclosure shows the benefits of reducing secondary interactions, which includes positively impacting chromatographic performance in terms of band broadening, peak tailing, and/or recovery which can then help increase resolution, peak capacity, and/or quantitative accuracy of liquid chromatography-based assays, particularly for liquid chromatography-based peptide mapping assays.

1. A method of separating and analyzing a metal-sensitive sample comprising:
injecting the metal-sensitive sample into a chromatographic system having a fluid-contacting coating on a metallic surface, wherein the fluid-contacting coating comprises an alkylsilyl;
flowing the injected metal-sensitive sample through the chromatographic system;
separating the metal-sensitive sample, wherein coating the metallic flow path of the chromatographic system reduces peak tailing; and
passing the separated metal-sensitive sample through a mass spectrometer to analyze the separated sample.

2. The method of claim 1, wherein peak tailing is reduced by at least about 50%.

3. A method of separating a metal-sensitive sample comprising:
providing a chromatographic system having a fluid-contacting coating on at least a portion of a metallic flow path;
injecting the metal-sensitive sample into the chromatographic system;
flowing the injected metal-sensitive sample through the chromatographic system;
separating the flowing metal-sensitive sample, wherein the metal-sensitive sample comprises a peptide; and
performing mass spectrometry on the separated metal-sensitive sample.

4. A method of separating a metal-sensitive sample comprising:
injecting the sample into a chromatographic system having a fluid-contacting coating on a metallic surface, wherein the fluid-contacting coating comprises an alkylsilyl;
flowing the metal-sensitive sample through the chromatographic system;
separating the metal-sensitive sample, wherein the metal-sensitive sample comprises a peptide; and
analyzing the separated metal-sensitive sample with a UV detector.

5. The method of claim 1, wherein the fluid-contacting coating increases recovery of the metal-sensitive sample by at least about 20%.

6. The method of claim 1, wherein the fluid-contacting coating does not substantially change retention of the metal-sensitive sample.

7. The method of claim 1, wherein the fluid-contacting coating does not result in peak loss or diminish recovery of the metal sensitive sample.

8. The method of claim 1, wherein the metal-sensitive sample does not bind to the fluid-contacting coating.

9. The method of claim 1, wherein the metal-sensitive sample is selected from the group consisting of glutamic acid and aspartic acid.

10. The method of claim 1, wherein the fluid-contacting coating comprises bis(trichlorosilyl)ethane or bis(trimethoxysilyl)ethane.

11. The method of claim 3, wherein providing the chromatographic system having the coating comprises assessing polarity of metal-sensitive compound; selecting a desired contact angle and coating material based on polarity assessment; and adjusting hydrophobicity of the flow path by vapor deposition of alkylsilyl.

12. The method of claim 3, wherein the fluid-contacting coating reduces peak tailing.

13. The method of claim 12, wherein peak tailing is reduced by at least about 50%.

14. The method of claim 4, wherein the fluid-contacting coating reduces peak tailing.

15. The method of claim 14, wherein peak tailing is reduced by at least about 50%.

16. The method of claim 3, wherein the fluid-contacting coating increases recovery of the metal-sensitive sample by at least about 20%.

17. The method of claim 4, wherein the fluid-contacting coating increases recovery of the metal-sensitive sample by at least about 20%.

18. The method of claim 3, wherein the metal-sensitive sample does not bind to the fluid-contacting coating.

19. The method of claim 4, wherein the metal-sensitive sample does not bind to the fluid-contacting coating.


SEQUENCE LISTING (15 sequences)

SEQ ID NO: 1 - protein, 22 aa, Artificial Sequence
  Description of Artificial Sequence Synthetic peptide
GFYPSDIAVEWESNGQPENNYK

SEQ ID NO: 2 - protein, 6 aa, Artificial Sequence
  Description of Artificial Sequence Synthetic peptide
PENNYK

SEQ ID NO: 3 - protein, 20 aa, Artificial Sequence
  Description of Artificial Sequence Synthetic peptide
VDNALQSGNSQESVTEQDSK

SEQ ID NO: 4 - protein, 20 aa, Artificial Sequence
  Description of Artificial Sequence Synthetic peptide
VSNALQSGSSQSSVTSQSSK

SEQ ID NO: 5 - protein, 20 aa, Artificial Sequence
  Description of Artificial Sequence Synthetic peptide
VENALQSGSSQSSVTSQSSK

SEQ ID NO: 6 - protein, 20 aa, Artificial Sequence
  Description of Artificial Sequence Synthetic peptide
VENALQSGSSQESVTSQSSK

SEQ ID NO: 7 - protein, 20 aa, Artificial Sequence
  Description of Artificial Sequence Synthetic peptide
VENALQSGSSQESVTEQSSK

SEQ ID NO: 8 - protein, 20 aa, Artificial Sequence
  Description of Artificial Sequence Synthetic peptide
VENALQSGSSQESVTEQESK

SEQ ID NO: 9 - protein, 20 aa, Artificial Sequence
  Description of Artificial Sequence Synthetic peptide
VDNAKQSGNSQESVTEQDSK

SEQ ID NO: 10 - protein, 12 aa, Artificial Sequence
  Description of Artificial Sequence Synthetic peptide
  MOD_RES (5)..(5): pTyr
  MOD_RES (9)..(9): pTyr
TRDIYETDYYRK

SEQ ID NO: 11 - protein, 23 aa, Artificial Sequence
  Description of Artificial Sequence Synthetic peptide
YPIVSIEDPFAEDDWEAWSHFFK

SEQ ID NO: 12 - protein, 10 aa, Unknown
  Description of Unknown Angiotensin I sequence
DRVYIHPFHL

SEQ ID NO: 13 - protein, 213 aa, Artificial Sequence
  Description of Artificial Sequence Synthetic polypeptide
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 449 aa, Artificial Sequence
  Description of Artificial Sequence Synthetic polypeptide
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 12 aa, Artificial Sequence
  Description of Artificial Sequence Synthetic peptide
  MOD_RES (6)..(6): pThr
VNQIGTLSESIK